(19) United States
(12) Patent Application Publication
(10) Pub. No.: US 2023/0290433 A1
(43) Pub. Date: Sep. 14, 2023

(54) VIRTUAL TRANSCRIPTOMICS

(71) Applicant: Elucid Bioimaging Inc., Boston, MA (US)

(72) Inventors: Andrew J. Buckler, Boston, MA (US); Ljubica Matic, Solna (SE); Ulf Hedin, Ronninge (SE)

(21) Appl. No.: 17/693,229

(22) Filed: Mar. 11, 2022

(51) Int. Cl. G16B 5/20

(52) U.S. Cl. CPC G16B 5/20

(57) ABSTRACT

Atherosclerotic plaque phenotyping by image data analysis, e.g., using conventional computed tomography angiography (CTA) or other imaging modalities can elucidate the molecular signature of atherosclerotic lesions on a per-patient basis.

### FEDERALLY SPONSORED RESEARCH OR DEVELOPMENT

This invention was made with Government support under Grant No. HL126224 awarded by the National Heart, Lung, and Blood Institute of the National Institutes of Health. The Government has certain rights in the invention.

### TECHNICAL FIELD

This invention relates to analyzing atherosclerotic plaques non-invasively, and more particularly to providing patient-specific morphological and transcriptomic information based on imaging data from a plaque.

### BACKGROUND

Cardiovascular disease (CVD) is the most common cause of death and disability in the world, mainly by myocardial infarction and ischemic stroke from unstable atherosclerosis,¹ which exerts an exorbitantly high financial burden to society.² Risk management of patients is largely dependent on population-based scoring methods such as the Framingham Risk Score or secondary prevention in patients with established disease^{3, 4} and development of diagnostics for more precise patient categorization is warranted. Despite discoveries of new predictive plasma biomarkers⁵ and improved plaque imaging,⁶ routine diagnostic methods for identification of individuals and lesions at high risk for atherothrombosis in coronary or extracranial arteries are still lacking.⁷ In addition, strategies to implement tailored, personalized pharmacotherapy remain limited without practical non-invasive assessment of biological and molecular disease features.⁸

Development of quantitative imaging biomarkers (QIBs) for guiding cancer therapy based on non-invasive imaging using molecular signatures from tissue biopsies as a truth basis, has been met with enthusiasm.^{26, 27} A similar approach is more challenging for CVD, as acquisition of plaque tissue biopsies from living patients is not generally practical.

### SUMMARY

The present disclosure relates to methods of determining atherosclerotic plaque (e.g., plaques found in the walls of various arteries, including without loss of generality, coronary arteries, carotid arteries, femoral arteries, aorta, etc.) molecular phenotype non-invasively, thereby providing a subject-specific predictive model for gene expression that we refer to herein as virtual transcriptomics. These methods were developed by training machine intelligence models to interpret conventional plaque image data, such as computed tomography angiography (CTA) image data, with paired global microarray-based transcriptomic analyses of vascular wall tissues. The results described herein demonstrate the feasibility of using non-invasive, commonly available imaging protocols combined with advanced morphological and molecular characterization of atherosclerotic plaques and machine intelligence methods to determine per-patient molecular level signatures, with potential for optimizing personalized therapy in the prevention of myocardial infarction and ischemic stroke.

In one aspect, the disclosure features methods of generating phenotypic data for an atherosclerotic plaque from a subject, the methods include, comprise, or consist of: (a) receiving a non-invasively obtained imaging dataset for an atherosclerotic plaque from a subject; (b) processing the non-invasively obtained imaging dataset with a virtual tissue model to obtain quantitative plaque morphology data; (c) processing the quantitative plaque morphology data with a virtual expression model to obtain estimated gene expression data for the plaque from the subject; and (d) predicting which gene transcript levels are elevated and which gene levels are decreased in the plaque from the subject as compared to gene expression in a subject without atherosclerosis, thereby generating phenotypic data for the atherosclerotic plaque from the subject.

In certain embodiments, the non-invasively obtained imaging dataset is a radiological imaging dataset. In some embodiments, the non-invasively obtained radiological imaging dataset is obtained by computed tomography (CT), dual energy computed tomography (DECT), spectral computed tomography (spectral CT), computed tomography angiography (CTA), cardiac computed tomography angiography (CCTA), magnetic resonance imaging (MM), multi-contrast magnetic resonance imaging (multi-contrast MRI), ultrasound (US), positron emission tomography (PET), intra-vascular ultrasound (IVUS), optical coherence tomography (OCT), near-infrared radiation spectroscopy (NIRS), or single-photon emission tomography (SPECT) diagnostic images or any combination thereof.

In some embodiments, quantitative plaque morphology data includes structural anatomy data and tissue composition data. For example, the structural anatomy data includes data relating to a level of any one or more of remodeling, wall thickening, ulceration, stenosis, dilation, or plaque burden. In certain embodiments, the tissue composition data includes data relating to a level of any one or more of calcification, lipid-rich necrotic core (LRNC), intraplaque hemorrhage (IPH), matrix, fibrous cap, or perivascular adipose tissue (PVAT).

In some embodiments, the gene transcript levels are based on gene transcripts whose expression profiles are illustrated in FIG. 5. In certain embodiments, the gene transcript levels are based on gene transcripts listed in Table 4. In some embodiments, the gene transcript levels are based on gene transcripts listed in Table 5.

In various embodiments, the method further includes using the predicted gene transcript levels for gene-set enrichment analysis to provide a patient-specific determination of one or more mechanisms related to the subject's plaque pathophysiology, plaque instability, or both.

In some embodiments, the one or more mechanisms related to plaque pathophysiology, plaque instability, or both include one or more of smooth muscle cell (SMC) proliferation, extracellular matrix (ECM) organization, collagen degradation, phospholipid efflux, degradation of the extracellular matrix, positive regulation of intracellular signal transduction, regulation of epithelial to mesenchymal transition, regulation of IGF transport and uptake, homotypic cell-cell adhesion, neutrophil mediated immunity, apoptotic process, regulation of protein ectodomain proteolysis, cholesterol efflux, chylomicron remnant clearance, response to laminar fluid shear stress, or neutrophil mediated immunity.

In certain embodiments, imaging data intensity is corrected to more closely represent the originally imaged plaque using a patient-specific three-dimensional point spread function.

In some embodiments, the virtual expression model includes a supervised continuous gene expression model. In the same or other embodiments, the virtual expression model includes a dichotomized gene expression model of gene expression levels above or below a median expression value.

In some embodiments, a plaque classified as having a high level of calcification compared to a reference level is predicted to have a high level of expression of proteoglycan 4 and a low level of expression of Speedy/RINGO Cell Cycle Regulator Family Member E1 as compared to corresponding reference levels of expression in a plaque that does not have a high level of calcification.

In some embodiments, a plaque classified as having a large LRNC compared to a reference level is predicted to have a high level of expression of matrix metalloproteinase 12 and a low level of expression of rap guanine nucleotide exchange factor 4 as compared to corresponding reference levels of expression in a plaque that does not have a large LRNC.

In certain embodiments, a plaque classified as having a high level of IPH compared to a reference level is predicted to have a higher level of expression of biliverdin reductase B and of cyclin-dependent kinase inhibitor 2A, and a lower level of expression of nodal modulator 1 as compared to corresponding reference levels of expression in a plaque that does not have a high level of IPH.

In some embodiments, a plaque classified as having a large amount of matrix compared to a reference level is predicted to have high level of expression of interleukin-13 and a low level of expression of Nudix Hydrolase 21 as compared to corresponding reference levels of expression in a plaque that does not have a large amount of matrix.

In certain embodiments, a plaque classified as having a high level of calcification compared to a reference level is predicted to have low level of expression of Solute Carrier Family 30 Member 1 and of Solute Carrier Family 39 Member 8 as compared to corresponding reference levels of expression in a plaque that does not have a high level of calcification.

In some embodiments, a plaque classified as having a high level of IPH compared to a reference level is predicted to have high level of expression of Solute Carrier Family 30 Member 1 and of Solute Carrier Family 39 Member 8 as compared to corresponding reference levels of expression in a plaque that does not have a high level of IPH.

In some embodiments, a plaque classified as having a large LRNC compared to a reference level is predicted to have high level of expression of Solute Carrier Family 39 Member 8 and low level of expression of Solute Carrier Family 30 Member 1 as compared to corresponding reference levels of expression in a plaque that does not have a large LRNC.

In certain embodiments, a plaque classified as having a large LRNC compared to a reference level is predicted to have high level of expression of IL1R1 as compared to a corresponding reference level of expression in a plaque that does not have a large LRNC.

In some embodiments, a plaque classified as having high level of calcification compared to a reference level and low level of IPH compared to a reference level is predicted to have high level of expression of TGFBR2 as compared to a corresponding reference level of expression in a plaque that does not have a high level of calcification and a low level of IPH.

In some embodiments, a low level of expression of MIR125B1 compared to a reference level is predicted in a plaque with a combined large LRNC and a high level of IPH, and a high level of expression pf MIR125B1 compared to a reference level is predicted in a small plaque with a high level of CALC compared to a reference level.

In certain embodiments, a low level of expression of MIR718 compared to a reference level is predicted in a small plaque with a high level of CALC, and a level of expression of MIR718 is increased in a larger plaque as a level of CALC decreases.

In some embodiments, a level of expression of MIR4536-1 is predicted to be lower in a large plaque with an increased level of CALC and is predicted to be even lower in a plaque with a decreased level of CALC.

The present disclosure provides advanced software-based techniques to extract data embedded in image data, which are otherwise not readily appreciated visually or quantitatively, to provide biomarkers to identify patients with unstable atherosclerosis plaques and imaging to localize such unstable atherosclerotic plaques. The new methods provide more accurate characterization of plaques to provide better clinical care, to enable the development of new drugs that are more effective for patients at risk of ischemic events due to unstable plaques, and to provide support for surgical interventions.

While data has been documented at individual scales, e.g., in vitro data at the cellular and molecular scale, microscopy data at histopathology scale, and radiological data at macroscopic scale, there is a dearth of linkages across these scales. As disclosed herein, applicant has discovered that biomarkers can go beyond indicating that there is a problem, to specifically categorize a patient's plaque and to recommend the best way to address the problem. Accordingly, the present invention fills gaps in understanding the extent and rate of progression of atherosclerosis by combining virtual tissue modeling and transcriptomics, to thereby provide recommendations for potential treatment alternatives.

As used herein a “reference level” is generally a level considered normal, i.e., neither under or over the level observed in healthy subjects. For example, a mean level of expression of a gene in a healthy individual can be considered a reference level for expression of a given gene. In addition, a reference level for a particular plaque is defined in terms of a particular morphology features known to be found in plaques. For example, a reference level for CALC or LRNC or the like as described herein, can be defined for any feature found in so-called “stable” plaques, in which case other levels, e.g., higher or lower, e.g., as found in, for example, “vulnerable” plaques, for a particular feature, can then be determined with respect to the reference level of that feature in a stable plaque.

As used herein, the articles “a” and “an” refer to one or to more than one (e.g., to at least one) of the grammatical object of the article.

The term “or” is used herein to mean, and is used interchangeably with, the term “and/or,” unless context clearly indicates otherwise.

“About” and “approximately” shall generally mean an acceptable degree of error for the quantity measured given the nature or precision of the measurements. Exemplary degrees of error are within 10 percent (%) of a given value or range of values.

As described herein, the terms “subject” or “patient” are used interchangeably and refer to a warm-blooded animal such as a mammal afflicted with a particular disease, disorder, or condition. It is explicitly understood that mice, rats, guinea pigs, rabbits, monkeys, cats, dogs, pigs, sheep, goats, horses, cattle, and humans are examples of subjects within the scope of the meaning of the term.

Additional definitions are set out throughout the specification.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

This patent or application file contains at least one drawing executed in color. Copies of this patent or patent application publication with color drawing(s) will be provided by the Office upon request and payment of the necessary fee.

FIG. 1 is a diagram showing the study workflow for plaque characterization using two levels of machine intelligence models to provide both quantitative plaque morphology as well as estimated tissue gene expression on a per-patient basis.

FIG. 2 is a diagram showing the multiple objectively validated measurements and characterizations that were made to were made to characterize plaque morphology by computed tomography angiography (CTA) analysis software.

FIGS. 3A-3E are processed 3-D images of the artery and lesion from the CTA obtained from an 86 year old white man with a transient ischemic attack.

FIGS. 4A-4D is an image showing the processing of a CTA image with the analytical software (ElucidVivo®).

FIG. 5 is a heatmap showing the highest associations between tissue characteristics resulting from unsupervised cluster analysis.

FIG. 6A is a representation of the performance of a continuous-value expression model in the form of a scatter plot with multiple regression lines, where each curve plots the best model fit for each of the different predictor sets considered for Solute Carrier Family 30 Member 1 (SLC30A1).

FIG. 6B is a table comparing the correlation for each of three tissue types on expression level for two transporter genes representing Zn influx (Solute Carrier Family 39 Member 8 (SLC39A8)) and Zn efflux (SLC30A1) (B; Pearson's r correlation shown to indicate relative direction rather than magnitude; MaxCALCArea, MaxIPHArea, and MaxLRNCArea indicates maximum cross-sectional area; Prop suffix indicates proportional occupancy of tissue relative to overall wall area).

FIG. 6C is a schematic showing that as Intra-Plaque Hemorrhage (IPH) increases and the level of calcified tissue (CALC) decreases, the expression of the two transporter proteins, SLC39A8 and SLC30A1, increases, but as the size of a Lipid-Rich and Necrotic Core (LRNC) increases, expression of an influx transporter protein, SLC39A8, increases and an efflux transporter protein, SLC30A1, decreases.

FIG. 6D is a representation of the performance of a continuous-value expression model in the form of a scatter plot with multiple regression lines, where each curve plots the best model fit for each of the different predictor sets considered for Transforming Growth Factor-beta (TGF-β) receptor type 2 (TGFBR2).

FIG. 7 is a schematic showing the differences observed in plaque morphology associated with increased expression of either TGFBR2 or Interleukin-1 receptor 1 (IL1R1). Higher TGFBR2 expression is associated with a high CALC burden, whereas IL1R1 is expressed more in plaques with a larger LRNC.

FIGS. 8A-8D are representative immunohistochemistry images showing staining for TGFBR2 (panels A and C) and IL1R1 (panel B and D) in plaques dominated by either a lipid-rich necrotic core (LRNC) or level of CALC.

FIG. 9A are receiver operating characteristic (ROC) curves for classifying MIR125B1 by various predictor sets, where classification performance differed based on the predictors sets used.

FIG. 9B are bar graphs showing that volumes for plaque tissue components typifying lower (below median) expression of each of the microRNAs demonstrated different distributions of the interrogated tissue types.

FIG. 9C are bar graphs showing that changes in plaque composition as expression increased show even greater differences, e.g., higher CALC levels predict lower expression of MIR125B1, but decreased expression of MIR718 and MIR4535-1.

FIG. 10 is a schematic showing the biological processes that were identified as being significantly determined by the robustly predicted transcripts.

FIG. 11 are heatmaps (left side) for four sequestered (unseen) test patients (T1-T4), representing: plaque morphology profile; predicted expression of the top 20 most significant predicted transcripts; and true expression of corresponding transcripts obtained from microarray analysis of carotid endarterectomy (CEA) specimens. Dominant mechanisms obtained from pathway analysis by single specimen gene-set enrichment analysis (GSEA) for each patient (right side).

### DETAILED DESCRIPTION

The present methods use image analysis techniques to obtain morphological data from image data for atherosclerotic lesions together with gene expression data obtained, for example, by microarrays or other suitable methods (such as RNA seq as another non-limiting example) as an objective truth basis of plaque biology to create computational models to predict molecular plaque signatures, determine plaque phenotype, and aid clinical decision making in patients without analysis of tissue specimens. Two levels of ground truth are used, one to support characterization of plaque tissue by radiology imaging, e.g., CTA, based on histology by microscopy from an independent tissue bank to create “virtual tissue models,” and a second one to quantify molecular mechanisms based on transcriptomics using “virtual expression models.” Resulting models were then deployed on previously unseen, non-invasive imaging data (hold-out patients) for which actual transcriptomics data was available for validation. These results support methods of predictive “virtual transcriptomics” on a per-patient basis.

General Methodology

The methods described herein include methods of generating phenotypic data for an atherosclerotic plaque from a subject. These methods include receiving a non-invasively obtained imaging dataset for an atherosclerotic plaque from a subject; processing the non-invasively obtained imaging dataset with a virtual tissue model to obtain quantitative plaque morphology data; processing the quantitative plaque morphology data with a virtual expression model to obtain estimated gene expression data for the plaque from the subject; and predicting which coding and non-coding transcript levels are elevated and which levels are decreased in the plaque from the subject as compared to gene expression in a subject without atherosclerosis, thereby generating phenotypic data for the atherosclerotic plaque from the subject.

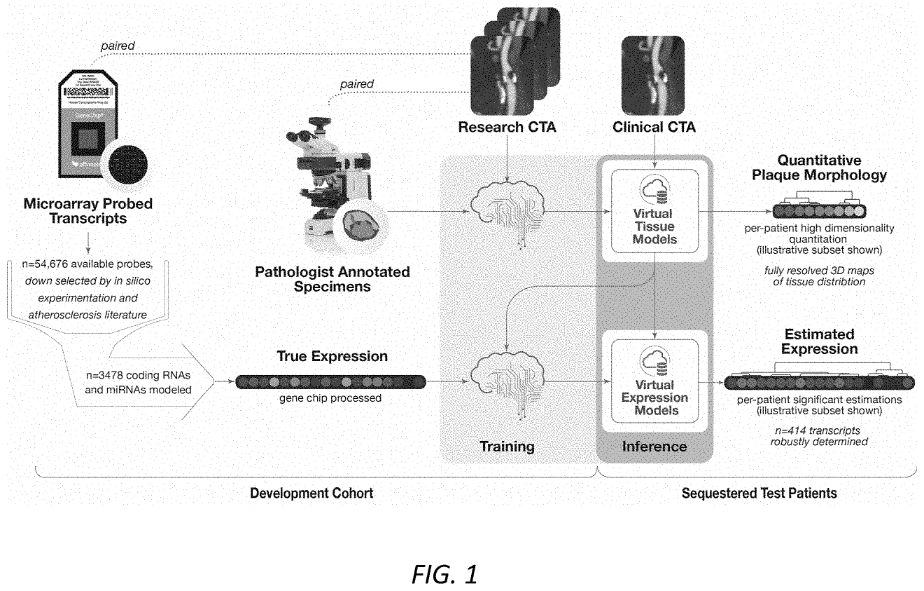

As shown in FIG. 1, there are two parts to the new methods. First, there is a development cohort, shown on the left side of the image, and then the research is applied to sequestered test patients, as shown on the right half of the image. This second aspect also applies to any new patient sample image data. For the development cohort, research CTA images and clinical CTA are fed into the modeling software. Tissue measurements made at the first level of processing include structural anatomy and tissue characterization, using virtual tissue modeling software, which were trained using pathologist annotated specimens, and generate quantitative plaque morphology data.

These data are then fed forward as inputs to the models to elucidate molecular profiles determining plaque phenotype. The plaques are characterized as having “high” or “low,” levels of calcification (CALC), lipid-rich necrotic core plaque (LRNC), intra-plaque hemorrhage (IPH), and matrix/fibrous tissue (MATX). “High” and “low” are based on median measurements obtained from the development/training set used in the tissue model software. In some instances, reference cohorts that cover a very large number of cases are managed, and then quartiles are identified for these and other measurands. The high and low is thereby more robustly determined, as being on the boundary of the 2nd to the 3rd quartile.

Once a plaque is profiled and established, the experimental workflow utilizes a set of cases with paired transcriptomic data from microarrays in a development cohort and subsequently in a cohort of sequestered test patients. These truth data were used to build the virtual expression models in the development cohort, then locked down for application to the sequestered test patients as a validation of model capability.

In a clinical setting, a clinician would undergo the following steps: a) obtain non-invasive images, e.g., through CTA, b) process the images against virtual tissue models to obtain quantitative plaque morphology data (which gives information about the profile, characterization, type of plaque); c) this information is further processed against one or more virtual expression models to obtain an estimated gene expression data for the plaque from the subject; d) this in turn allows the clinician to predict which gene transcript levels are elevated and which gene levels are decreased in the plaque, which would also give information about the mechanisms related to plaque pathophysiology, plaque instability, or both, thereby generating phenotypic data for the atherosclerotic plaque from the subject. The clinician is then able to determine the best treatment plan for the subject.

Non-Invasive Imaging

A non-invasively obtained imaging dataset, i.e., image(s) of the plaques in arteries, can be obtained by various methods that are well known in the art. In some embodiments, the imaging dataset is obtained by radiological methods. For instance, any of the following can be employed: computed tomography (CT), dual energy computed tomography (DECT), spectral computed tomography (spectral CT), computed tomography angiography (CTA), cardiac computed tomography angiography (CCTA), magnetic resonance imaging (MM), multi-contrast magnetic resonance imaging (multi-contrast MRI), ultrasound (US), positron emission tomography (PET), intra-vascular ultrasound (IVUS), optical coherence tomography (OCT), near-infrared radiation spectroscopy (NIRS), or single-photon emission tomography (SPECT). In a particular embodiment, CTA is utilized.

For example, in one embodiment, CTA can be performed as a pre-operative routine procedure in the hospital using site-specific image acquisition protocols. CTA exams can be performed with 100 or 120 kVp, variation of CTDIvol 16 cm between 13.9 and 36.9 mGy or CTDIvol32 cm 7.9-28.3 mGy. Contrast injection rates and amounts followed by a saline chaser can be used as required. In general, a caudocranial scanning direction can be selected from the aortic arch to the vertex, using intravenous contrast. An axial image reconstruction of about 0.5 to about 1.0 mm, e.g., 0.650 mm, 0.9 mm, or 1.0 mm can be used, and transferred into a digital workstation for vascular CTA image analysis. Variations of this example are envisioned and would be appreciated by one of skill in the art.

Virtual Tissue Models

Images obtained from the non-invasive imaging methods described herein are loaded into an image processing software, e.g., ElucidVivo® (Elucid Bioimaging Inc., Boston, Mass.) software,^{40, 41, 42, 43, 34} which outlines (segments) the luminal and outer wall surfaces of the common, internal, and external carotid arteries to provide quantitative plaque morphology data. See also, U.S. Pat. Nos. 10,176,408, 10,740,880, 11,094,058, and 11,087,460, each of which is incorporated herein by reference. Specifically, the software creates fully 3-dimensional segmentations of lumen, wall, and each tissue type at an effective resolution ≈3× higher than the reconstructed voxel size with improved soft tissue plaque component differentiation relative to manual inspection. The common and internal carotid artery are defined as a target with lumen and wall evaluated automatically and, when needed, edited manually.

The software provides vessel structure measurements including the degree of stenosis (calculated both by area or diameter), wall thickness (distance between the lumen boundary to outer vessel wall boundary), and remodeling index (the ratio of vessel area with plaque to a vessel area without plaque used as reference). Investigations in animal models and histological analyses of human plaque lesions have characterized distinct, but common, structural and biological tissue characteristics such as enhanced inflammation, accumulation of a large lipid-rich and necrotic central core (LRNC), intra-plaque hemorrhage (IPH), matrix/fibrous tissue, a thin and rupture-prone fibrous cap from extracellular matrix (ECM) degradation, apoptosis of smooth muscle cells (SMCs), and level of calcification (CALC).³⁰ More recently, the morphological and biological features of atherosclerotic plaques in humans have also been corroborated by molecular pathway analyses of the human plaque transcriptome.^{31, 32}

The software includes algorithms to decrease blur caused by image formation in the scanner. A patient-specific 3-dimensional point spread function is adaptively determined so that image intensities are restored to represent the original materials imaged more closely, which mitigates artefacts such as calcium blooming, and enables discrimination of less prominent tissue types. In particular, the image restoration is undertaken in concert with tissue characterization based on expert-annotated histology, e.g., as described in U.S. Pat. Nos. 10,176,408, 10,740,880, 11,094,058, and 11,087,460, each of which is incorporated herein by reference.

The overlapping densities of tissues such as LRNC and IPH necessitate a method for accurate classification. To avoid limitations of conventional analysis of CTA utilizing fixed thresholds, the accuracy required for elucidating molecular pathways was achieved by algorithms that account for distributions of tissue constituents rather than assuming constant material density ranges. In this way, the software makes mathematical judgments to interpret the Hounsfield units (HU) of adjacent voxels by maximizing criteria that mimic expert annotation at microscopy, simultaneously mitigating variation between scanners, reconstruction kernels, and contrast levels. In this way, the software fundamentally addresses subjectivity intrinsic to other analysis methods.

Processing the non-invasively obtained image data with the virtual tissue models provides output information relating to quantitative plaque morphology, such as structural anatomy data and tissue composition data. For example, structural anatomy data includes measuring any one or more of the following in the lumen and wall: remodeling, wall thickening, ulceration, stenosis, dilation, plaque burden, or any of the measurands listed in the Table 1 below.

As outlined in Table 1, vessel structure measurements included the degree of stenosis (calculated both by area or diameter), wall thickness (distance between the lumen boundary to outer vessel wall boundary), and remodeling index (the ratio of vessel area with plaque to a vessel area without plaque used as reference).

TABLE 1
Structural Calculations of Vessel Anatomy

| Measurand | Description | Type and Units |
|---|---|---|
| Lumen Area | Cross-sectional area of blood | mm² |
| | channel along the vessel centerline | |
| % Stenosis | (1 - ratio of minimum lumen with | % |
| (Max | plaque to reference lumen without | |
| Stenosis) | plaque) ×100, both by area and by | |
| | diameter | |
| Wall Area | Cross-sectional area of vessel | mm² |
| | minus the Lumen Area along the | |
| | vessel centerline | |
| Wall | Maximum cross-sectional wall | mm |
| Thickness | thickness along the vessel centerline | |
| Max Wall | Largest value of the wall thickness | mm |
| Thickness | | |
| Plaque Burden | Wall Area/(Wall Area + Lumen Area) | unitless ratio |

Tissue composition data included calcification (CALC), lipid-rich necrotic core plaque (LRNC), intra-plaque haemorrhage (IPH), and matrix/fibrous tissue (MATX), see Table 2 below.

TABLE 2
Calculations of Tissue Characteristics

| Measurand | Biological Evidence on Histopathology |
|---|---|
| Calcification | intimal/medial spaces with evidence of calcium |
| | primarily in the form of hydroxyapatite |
| | osteoblasts or osteoid present in above spaces |
| | no appreciable lipid or necrotic tissue in above spaces |
| Lipid-rich | lipid droplets intermixed ECM (appear clear due to |
| Necrotic | removal) |
| Core | necrotic amorphous eosinophilic material |
| (LRNC) | acellular |
| | often surrounded by fibrotic tissue generated by smooth |
| | muscle cells/fibroblasts |
| | lack of microvasculature |
| Intra-plaque | erythrocytes in the deeper regions of the plaque |
| Hemorrhage | with or without communication to lumen or |
| (IPH) | neovasculature |
| | Fresh: RBC is intact and unorganized |
| | Recent (5+ days): inflammatory response organizes |
| | the RBC via hemolysis, fibroblast activity, |
| | macrophage activity |
| Matrix | Note elongated striated appearance which describe: |
| | intimal meshwork of dense or loose, homogeneous/ |
| | organized collagen ECM (appear striated) |
| | embedded smooth muscle cells/fibroblasts (note |
| | elongated nuclei) |
| | no appreciable lipid or necrotic tissue |
| | may have microvasculature |

Volume measurements, either in place of or additive to area measurements can also be utilized. Likewise, various forms of spatially labelled data that represent these may also be used.

Virtual Expression Models

The virtual expression models are built from a variety of machine learning models. For example, such as those described in U.S. Pat. Nos. 10,176,408, 10,740,880, 11,094,058, and 11,087,460. Briefly, any of several methods, devices, and/or other features which are used to perform a specific informational task (such as classification or regression) using a limited number of examples of data of a given form, and are then capable of exercising this same task on unknown data of the same type and form. The machine (e.g., a computer or processor) will learn, for example, by identifying patterns, categories, statistical relationships, etc., exhibited by training data. The result of the learning is then used to predict whether new data exhibits the same patterns, categories, and statistical relationships. Examples of such models include neural networks, SVMs, decision trees, hidden Markov models, Bayesian networks, Gram Schmidt models, reinforcement-based learning, genetic algorithms, and cluster-based learning. Multiple methods may be used to create the pool of trained machines from which the choice is made. These can include methods of feature selection and reduction, ranking of features, random generation of feature sets, correlations among features, ICA and PCA, parameter variation, and any methods known to those skilled in the art.

Supervised learning occurs when training data is labelled to reflect the “correct” result, i.e., that the data belongs to a class or exhibits a pattern. Supervised learning techniques include neural networks, SVMs, decision trees, hidden Markov models, Bayesian networks, etc. Test data sets encompassing known class(es) can be used to determine if a trained learning machine is able to identify patterns in data and/or classify data. The test data set is preferably generated independently from the training data set. Training Data sets (of known or unknown classes) are used to train a learning machine. Regardless of whether the class of the data is known or unknown, the data can be adequate for training a learning machine. Unsupervised learning occurs when training data is not labelled to reflect the “correct” result, i.e., there is no indication within the data itself as to whether the data belongs to a class or exhibits a pattern. Unsupervised learning techniques include Gram Schmidt, reinforcement-based learning, cluster-based learning, etc.

In the present disclosure, collected tissue specimens were analyzed by Affymetrix microarray with 54,676 probes. Both supervised and unsupervised, as well as single variable and multi variable, methods were performed to assess the ability of non-invasive morphological measurements to identify dominant molecular mechanism using ex vivo transcriptomics in paired specimens as ground truth.

Analysis of correlation among morphology measurements followed by unsupervised clustering of those found to be relatively independent was performed to give a rough sense for relationships among morphology measurements and expression level. Pearson correlation was plotted qualitatively, and those with values less than 0.8 were assessed using a Euclidean distance function and hierarchically clustered according to the complete linkage method on both morphology measurement features and on samples comprising patient lesions, plotted as a heatmap. Single variable analysis was performed demonstrating the relationships between individual features and categoric classification for low vs. high expression (using the species-dependent median as cutoff), as well as for the specific expression level used as a continuous variable.

Multiple variable models were built and evaluated both for categoric classification for and separately for continuous variable estimation. A range of model types were built as, in general, we did not know which type would best fit the data a priori. The models were built and optimized using a variety of applied predictive modeling techniques, including averaged neural networks, support vector machines, linear regression with recursive feature elimination, partial least squares, and tree-based models. By way of example, the best performing categorical models were often artificial neural networks (ANNs). Feature selection in ANNs occurs by virtue of optimizing the value of coefficients applied on measurements to “hidden” units, and then from these hidden units to the output nodes, which express the output as class probabilities. In particular, we used an averaged neural network (avNNet). Often the best performing continuous value estimation models were least squares regression models with a form of regularization to optimize the tradeoff between bias and variance called ridge regression. Optimization in these models occurs by determining iterating over values of λ, where low values favor low bias to higher values that allow successively higher values of bias in the hopes of reducing variance.

All model types, including the two example types explained here, were implemented using the Caret package in R, with three levels of variation: first, by using differing sets of morphological measurements according to hypothesized physiological rationale confirmed by the unsupervised clustering; second, for each set, automated optimization using 10-fold cross validation (with two repeats) while simultaneously varying different tuning parameter values appropriate to the model type. Data was partitioned such that a training set on which the cross-validation was performed and a sequestered validation data set to test performance on unseen data after locking models down, in a 2 to 1 ratio at random by patient (training to sequestered). The cross-validation technique has been widely recognized as means to mitigate overfitting, and the sequestered partition was utilized to establish generalizability to at least one independent set. Models were selected after cross-validation by optimizing the area under the receiver operating characteristic curve (AUC/ROC) multiplied by Kappa for categoric classification, and the cross-correlation coefficient (CCC) multiplied by slope of the regression line for continuous valued estimation. The best performing models were selected and locked down before application to the sequestered data.

The models described herein are capable of identifying a number of transcripts (both coding and non-coding). Further, the models described herein can be altered or enhanced as deemed necessary by one of skill in the art. Additionally, microarrays are only one embodiment and other technologies can be utilized to obtain expression data (e.g., RNAseq, and other suitable technologies). Thus, it is understood that the Tables of data, e.g., Tables 4, 5, and 6 herein, provide examples of data, and the expression of additional genes is contemplated to be within the scope of the present application.

Generating Phenotypic Data for Atherosclerotic Plaques

The quantitative plaque morphology data (which relates to the profile, characterization, type of plaque) received from the virtual tissue model, as described in the section “Virtual Tissue Models” above, is processed against one or more virtual expression models, as described in section “Virtual Expression Models” above, to obtain estimated/predicted gene expression data for the plaque from the subject. In other words, the imaging models are further modeled against known gene-expression patterns (that is, the tissue models based on the imaging data are correlated to gene-expression patterns) to generate a predicted virtual expression model(s). The virtual expression models then in turn allows the clinician to predict which gene transcript levels are likely elevated and which gene levels are likely decreased in the plaque. Levels of gene expression (elevated/decreased/unchanged) are in reference to a non-atherosclerotic patient. This would then also give information about the mechanisms related to plaque pathophysiology, plaque instability, or both, thereby generating phenotypic data for the atherosclerotic plaque from the subject. The clinician is then able to determine the best treatment plan for the subject.

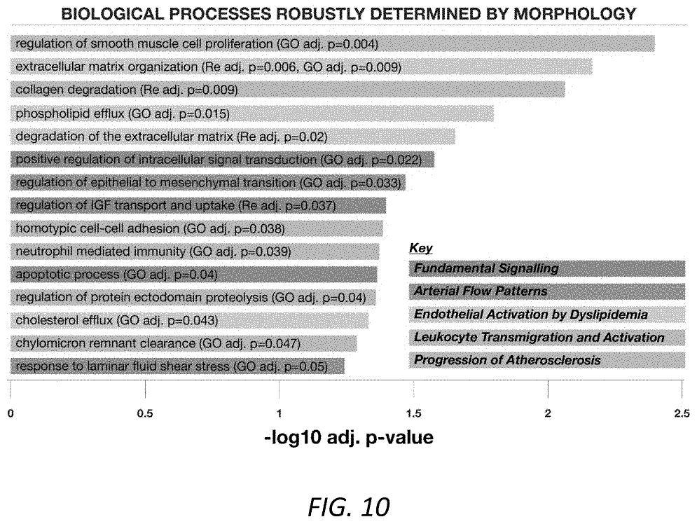

For example, it is known that there are several fundamental processes related to the pathophysiology of atherosclerosis and plaque instability, such as but not limited to, SMC proliferation; ECM organization; collagen degradation; apoptosis, phospholipid and cholesterol efflux; regulation of epithelial to mesenchymal transition, and neutrophil mediated immunity (or any of the processes outlined in FIG. 10). The virtual expression model obtained from the plaque of a patient provides information related to which genes are likely to be dysregulated. The genes that are predicted to be dysregulated can then provide information relating to which process or processes related to the pathophysiology of atherosclerosis and plaque instability might be affected. With this information, a treating clinician can then provide a suitable and targeted therapy that is specific to that particular patient.

To identify what is “picked up on” by the major categories of tissue morphology, we identified ranked lists of species for which robust determination is made of for each tissue category, according to variable importance of best-fit models. To form the list, the relative variable importance is multiplied by the dichotomized model AUC and Kappa, resulting in a ranking by gene reflective of the importance of the given tissue type in robust prediction.

We then evaluated in a discovery run, comprised of both unsupervised exploratory data analysis and supervised predictive modeling, which found 414 species could be predicted robustly using a cutoff value formed by multiplying the area under the receiver operating characteristic curve (AUROC) times Kappa, the former as a measure of the net classification performance, but augmented by the latter which ensures adequate performance in both classes (high and low expression in this case).

Selection of species eligible for pathway analysis was based on high values for AUC and Kappa (FIG. 8), as evidenced by ranking according to the product of point estimates against a cutoff of 0.4 (e.g., as obtained for AUC=0.8 and Kappa=0.5). This gene set was submitted to EnrichR (https://amp.pharm.mssm.edu/Enrichr/), further passing results from GO Biological process 2018 with adjusted p-values <0.05 to Revigo (http://revigo.irb.hr/) to determine non-duplicative processes, and finally merged with Reactome 2016 pathways that fell in the same range of significance.

### Examples

The invention is further described in the following examples, which do not limit the scope of the invention described in the claims. Additionally, Buckler et al., “Virtual Transcriptomics: Noninvasive Phenotyping of Atherosclerosis by Decoding Plaque Biology From Computed Tomography Angiography Imaging. Arterioscler Thromb Vasc Biol. 2021 May 5; 41(5):1738-1750. doi: 10.1161/ATVBAHA.121.315969. Epub 2021 Mar. 11. PMID: 33691476; PMCID: PMC8062292, and all supplementary data are incorporated herein by reference in their entireties.

In this study, we aimed to decode atherosclerotic plaque molecular phenotype non-invasively, making a predictive model for gene expression that we refer to as virtual transcriptomics. Our approach was focused on training machine intelligence models to interpret conventional CTAs with paired global microarray-based transcriptomic analyses of CEAs utilizing an established human biobank. The study demonstrates the feasibility of using non-invasive, commonly available imaging protocols combined with advanced morphological and molecular characterization of atherosclerotic plaques and machine intelligence methods to determine per-patient molecular level signatures, with potential for optimizing personalized therapy in the prevention of myocardial infarction and ischemic stroke. All data described herein was corrected for age and sex.

### Materials and Methods Used for Examples 1-5 Described Below

Human Samples and Plaque Tissue Transcriptomics

A total of 44 patients (40 development, 4 sequestered test) undergoing stroke-preventive CEA for high-grade (>50% NASCET³⁶) carotid stenosis were used in this study. Patients with high vs. low calcified carotid lesions on CTA were selected as previously described³⁷ and the study cohort demographics summarized in Table 3 below.

TABLE 3
Cohort Characteristics

| | Development set | Hold out set |
|---|---|---|
| Predictor | (n = 40) | (n = 4) |
| Age | 71.18 | 76.75 |
| S-Creatinine | 85.00 | 152 |
| S-CRP | 3.27 | 19.89 |
| HbA1c | 4.60 | 5.7 |
| Hb | 140 | 117 |
| LPK | 7.17 | 8.98 |
| S-Cholesterol | 4.39 | 3.48 |
| HDL | 1.27 | 1.05 |
| LDL | 2.38 | 1.75 |
| S-TG | 1.45 | 1.45 |
| Fibrinogen | 3.51 | 4.2 |
| SBP | 142 | 141 |
| DBP | 76 | 70 |
| Weight | 77.11 | 70.5 |
| BMI | 26.24 | 22.8 |
| Male | 72.5% (29) | 100% (4) |
| Hypertension treatment | 80% (32) | 100% (4) |
| Lipid lowering treatment | 95% (38) | 75% (3) |
| Smoker Y/N | 17.5% (7) | 25% (1) |
| Diabetes | 22.5% (9) | 25% (1) |

Briefly, CEAs were collected at surgery and retained within a biobank, details of sample collection and processing, and transcriptomic analyses by Affymetrix microarrays were as previously described.^{38, 39} Briefly, plaques were divided transversally at the most stenotic part; the proximal half of the lesion was used for RNA preparation while the distal half was fixed in 4% formaldehyde and prepared for histology. The microarray dataset is available from Gene Expression Omnibus (GSE125771). All samples were collected with informed consent from patients and the study was approved by the Ethical Review Board.

### CTA Image Analysis

Carotid CTA exams from the aortic arch to the vertex were performed with 100 or 120 kVp, variation of CTDIvol16 cm between 13.9-36.9 mGy or CTDIvol32 cm 7.9-28.3 mGy with intravenous contrast administered as previously described.³⁷ Axial image reconstruction of 0.625 mm were obtained and transferred for image analysis performed by E.K, blinded to histological and biochemical analysis. The ElucidVivo® (Elucid Bioimaging Inc., Boston, Mass.) software^{32, 34, 40-43} was used to provide characterization of plaque morphology. The software creates fully 3D segmentations of lumen, wall, and each tissue type at an effective resolution approximately 3× higher than the reconstructed voxel size with improved soft tissue plaque component differentiation relative to manual inspection. The common and internal carotid artery were defined as a target with lumen and wall evaluated automatically and, when needed, edited manually. The external carotid artery was excluded and image analysis limited to the proximal half of the lesion, corresponding to the tissue used for RNA isolation and microarray analysis.

The vessel wall was analyzed defining the plaque into different components: LRNC, CALC, IPH, matrix (MATX; representing plaque tissue not belonging to the other types), perivascular adipose tissue (PVAT), cap thickness (the smallest distance from LRNC to the lumen), and degree of stenosis. The software included algorithms to decrease blur caused by image formation in the scanner. A patient-specific 3D point spread function was adaptively determined so that image intensities were restored to represent the originally imaged materials more closely, which mitigated artefacts such as calcium blooming, and enables discrimination of less prominent tissue types.

The image restoration was undertaken in concert with a novel method for tissue characterization based on expert-annotated histology. The overlapping densities of tissues such as LRNC and IPH necessitated a method for accurate classification. To avoid limitations of conventional analysis of CTA utilizing fixed thresholds, the accuracy required for elucidating molecular pathways was achieved by algorithms that account for distributions of tissue constituents rather than assuming constant material density ranges. In this way, the software made mathematical judgements to interpret the Hounsfield units (HU) of adjacent voxels by maximizing criteria that mimic expert annotation at microscopy, simultaneously mitigating variation between scanners, reconstruction kernels, and contrast levels. In this way, the software fundamentally addressed subjectivity intrinsic to other analysis methods.

Analytic performance of the software was undertaken both for tissue composition accuracy relative to histopathology³⁴ and reader repeatability and reproducibility.⁴⁴

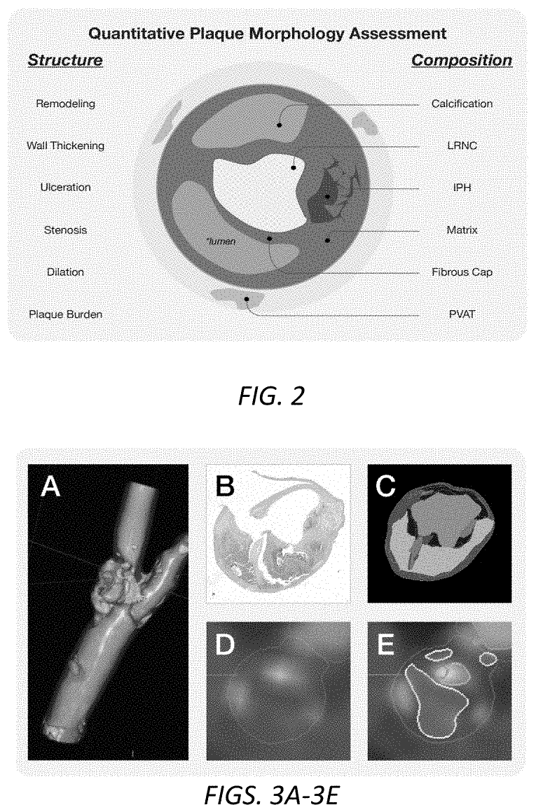

As shown in FIG. 2, multiple objectively validated measurements and characterizations were made to characterize plaque morphology by the CTA analysis software. These assessments included structural anatomy (“structure”) and tissue characterization (“composition”). (LRNC=lipid-rich necrotic core, IPH=intra-plaque hemorrhage, PVAT=perivascular adipose tissue). Analysis of carotid plaque tissue composition in the bifurcation of the left carotid artery in an 86 year old white man with a transient ischemic attack.

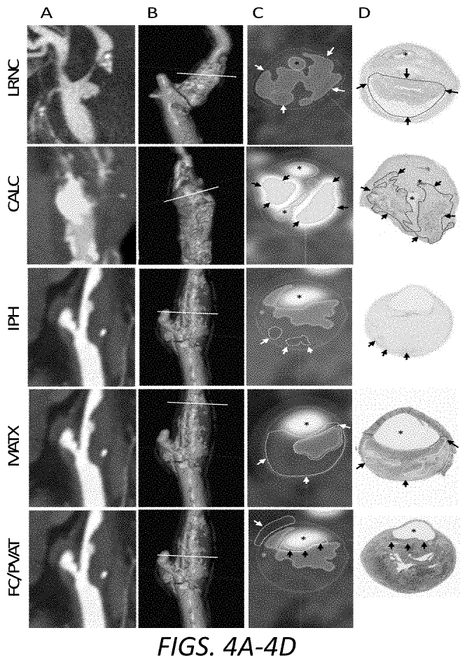

FIGS. 3A-3E show processed 3-D image of the artery and lesion from the CTA (FIG. 3A; common carotid artery partition—red (all colors are shown in a corresponding U.S. Patent Application and in Buckler et al., Arterioscler. Thromb. Vasc. Biol., 41(5):1738-1750 (2021), and all supplementary data, which are all incorporated herein by reference in their entireties); internal carotid—chartreuse; external carotid artery—purple); Histological section of the CEA specimen just distal of the bifurcation (FIG. 3B); section annotated by a pathologist indicating presentation of CALC (green) and LRNC (yellow). Cross sectional CTA image positioned near the histological section with outer wall and lumen segmented by the software with tissue characterization suppressed to show raw imagery (FIG. 3C) (here, MATX is shown with two colours to reflect that the pathologist marked dense fibrosis in dark blue and remaining MATX elements are indicated as a dark grey); the wall outline on CTA (FIG. 3D); the software's characterization of tissue composition (FIG. 3E). Note that only those tissue types present in a given sample appear; in this example, IPH and PVAT are not present. Colours in panels A-E: yellow—LRNC; aquamarine—CALC; light green—outer vessel wall boundary; orange—lumen boundary; dark blue—fibrotic tissue; blue—MATX.

FIGS. 4A-4D show processing of carotid CTA (4A) with analytical software (ElucidVivo®) demonstrating 3D image (4B), axial view of plaque (4C; white line in B indicates position of section; yellow LRNC) and corresponding histological section (4D) stained with Hematoxylin (LRNC, CALC), Perl's blue (IPH; arrows) and Masson's Trichrome to visualize fibrous tissue (MATX and FC). *, lumen; dashed lines and arrows mark tissue components; perivascular adipose tissue (PVAT) outlined (C; white arrow), not included in the histological sections of endarterectomy specimens.

### Predictive Modeling

Of the 54,676 probes for coding and non-coding RNAs represented in the microarray, 3478 probes were selected as most relevant to atherosclerosis based on the following criteria. First, we selected genes that were found to be highly dysregulated in comparisons of lesions with differing levels of calcification.³⁷ Briefly, global gene expression analysis comparing high vs. low calcified plaques (30-65% of plaque area vs. 0-2%) resulted in 3387 significantly differentially expressed probe-sets, of which 1783 were upregulated and 1604 downregulated (of total 70526 microarray probe sets, Bonferroni adjusted p<0.05). We then selected transcripts previously documented as being dysregulated in plaque instability³³ as well as those identified in a systems biology survey of atherosclerotic mechanisms,⁴⁶ adding a net of 91 additional transcripts identified in the cited works by symbol that were not already contained in the experimentally determined 3387.

Single variable analysis was performed to explore the relationships between individual measurements. Tables 1 and 2 summarize the investigated parameters. Categoric classification (low vs. high using the transcript-dependent median as cut-off), and continuous variables for specific expression level have been used.

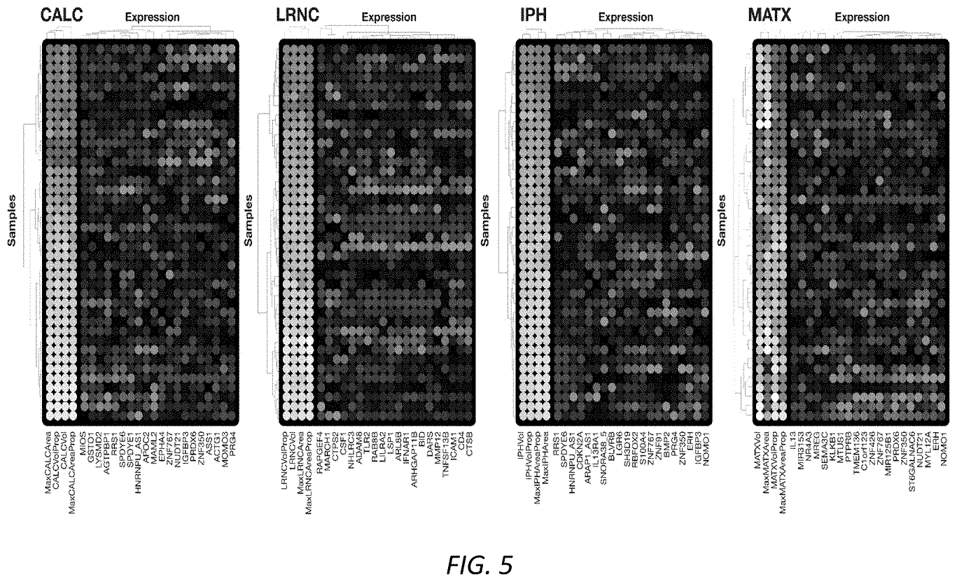

In addition, multiple variable analyses were performed, in the form of one set of models for dichotomized expression level as a categoric response variable and continuous-valued expression as a response variable. Four predictor sets were investigated (morphology predictors alone, clinical predictors alone, a combination of morphology, clinical and demographic predictors, and stenosis as a baseline); FIGS. 4 and 5 shows the relative performance in a given transcript. “Morphology” refers to a vector of plaque structure and tissue characteristic measures. “Clinical” refers to clinical variables such as body mass index (BMI), vital signs such as blood pressures, and serum biomarkers including for example C-reactive protein (CRP), cholesterol levels, and other recommended markers for patients with known or suspected cardiovascular disease. “Morphology+Clinical+Demog” combines the two sets as well as age and sex. “Stenosis” refers to the maximum measured degree of luminal narrowing.

A range of models including artificial neural networks (ANNs), support vector machines (SVMs), linear regression, partial least squares, and tree-based models were built to explore their performance to fit the data. The best performing categorical models were often ANNs, where feature selection occurs by virtue of optimizing the value of coefficients applied on measurements to hidden units, and then from these hidden units to the output nodes, which express the output as class probabilities. Least squares regression models often performed best for continuous value estimation, using ridge regression to optimize the trade-off between bias and variance. The physiological interpretability of the models was facilitated by use of the histologically validated inputs.

### Model Tuning Grids

Models were augmented as follows (all steps being taken before being locked down for application to the sequestered patient data):
1. Predictor sets were modelled including plaque morphology alone, clinical (i.e., serum biomarkers) alone, stenosis degree, and the combination across these. These are referred to as predictor sets.
2. The plaque morphology predictors were further varied by candidate mechanistic rationales. That is, it is important for the models to be biologically plausible, rather than a (only) performing algorithmic methods to select features, as means to introduce overfitting, increase generalizability, and to avoid the notion of the models being a “black box” that lacks an understanding of why it works, rather, that the models are supported by plausible biophysiological interpretation.
3. Next, given that the interrelationships between predictors and the response are generally complex, different model types were used, e.g., linear and logistic regression, penalized models, partial least squares, tree-based models, neural networks, and support vector machines.
4. Each model was iterated in cross-validation over a “tune grid” that varies internal parameters according to model type as follows: the dataset is split into 10 groups at random. Each group is taken in turn as a hold out set, with the other groups being used for model training, retaining the performance but discarding the model.
5. The terminology of train vs. test within cross-validation is not to be confused with the strict sequestration of data never included until the process completes.

Each model result was output to tabulate the highest-achieved performance on a transcript-by-transcript basis. Predictive performance was determined based on the accuracy of the prediction relative to the true value in each of the 3478 transcripts.

All models were built with three levels of variation: (i) differing sets of morphological measurements according to hypothesized physiological rationale (on all 3478 transcripts); (ii) automated optimization using 10-fold cross validation while simultaneously varying tuning parameter values (on all 3478 transcripts); and (iii) data was partitioned such that a training set on which the cross-validation was performed was strictly separated from a sequestered validation data set to test performance using locked-down models. Use of histologically validated plaque features produces interpretable models,⁴⁷ coupled with cross-validation, mitigated overfitting.

### Example 1: Relationships of Supervised and Unsupervised Models with Plaque Morphology and Gene Expression

Supervised and unsupervised statistical analytic methods were applied to assess the ability of CTA morphological measurements to identify molecular mechanisms obtained from transcriptomics of paired CEA specimens.

### Materials and Methods

Statistical Analysis

Unsupervised clustering was used to provide a rough sense for relationships between plaque morphology and expression levels. The hierarchical clustering is represented as a dendrogram split at points with Pearson correlation less than 0.8 using a Euclidean distance function according to the complete linkage method on both plaque morphology measurement features and on expression levels, plotted as a heatmap.

Plaque morphology data from image data analysis of CTAs in 40 patients was tested against gene expression levels for 3478 selected transcripts generated 414 transcripts meeting the MQ criteria for robustly predicted by plaque morphology, and subsequently subjected to unsupervised clustering (FIG. 5 and Table 4, below).

FIG. 5 shows the highest associations between tissue characteristics resulting from unsupervised cluster analysis. Plaque morphology is designated in shades of purple, darker indicating higher measured values and lighter indicating lower, in each of four quantitative measurands. MaxMATXArea is the largest cross-sectional area of the indicated tissue type, MaxMATXAreaProp is the corresponding proportional occupancy, MATXVol is the volume counterpart, and MATXVolProp denoting the proportional occupancy calculated on a volume basis. Transcript levels indicated in a scale with green indicating highest, red lowest, and black intermediate.

Table 4 below shows a list of gene transcripts that were determined to be either up or down regulated.

TABLE 4
Individual Species Robustly Determined by Tissue Type
Robustly Determined Species, by Tissue Type (whether up or down regulated)

| CALC | | LRNC | IPH | MATX | | |
|---|---|---|---|---|---|---|
| DLC1 | APOE | ARAP1_AS1 | RRS1 | PRRT3_AS1 | ABCA1 | OGN |
| SPDYE1 | MIR4536_1 | NHLRC3 | ZNF767 | ICAM1 | GTF3A | XIAP |
| MMP8 | PRG4 | ICAM1 | HNRNPU_AS1 | IGF2BP2 | RAB8B | DUSP19 |
| MIR142 | IL13 | ATG5 | SPDYE6 | ST6GALNAC6 | ZNF767 | NR4A3 |
| NUDT21 | BID | SLCO2A1 | SNORA38_5 | ADAM8 | ARL8B | SNAPC1 |
| PRDX6 | DDR1_1 | AGTPBP1 | IL4I1 | PSMD14 | CNN3 | CTSL1 |
| ZFYVE16 | XIAP | VAMP2 | AKT1 | SPDYE2 | PDGFRB | ATG3 |
| ZDHHC6 | ITIH4 | IL17B | ENG | GMDS | NOMO1 | MMP7 |
| LYSMD2 | ERH | TRAF5 | CXCR2P1 | DRAM1 | TLR2 | PRDX6 |
| LRP12 | WDR26 | NFE2L2 | GATM | ZNF350 | DSC2 | FCER1A |
| MMP7 | FAM35A | CSF1 | ZNF350 | LDB2 | FTO | B3GNT2 |
| HNRNPU_AS1 | NOMO1 | ADAM8 | ZNF91 | EGLN3 | TMEM136 | TANK |
| PKHD1L1 | MMP2 | ARHGAP18 | EPB41L3 | CR1 | PLEKHO1 | FAM134B |
| AGTPBP1 | SMARCC2 | UBE2D1 | IL13RA1 | ZDHHC6 | ZFAND5 | NFIL3 |
| ACTG1 | ARHGAP11B | DDX24 | SNORD97 | GCA | GLRX5 | NUDT21 |
| RRS1 | HIP1 | CTSB | IGFBP3 | TMOD1 | PTPRB | MIR142 |
| ZNF767 | APOC1 | IFNAR1 | APOC2 | ARHGAP11B | C1orf123 | IL10 |
| OR9A2 | TLR2 | MMP12 | TGFB1 | SLC36A1 | TLR7 | UNC5C |
| DRAM1 | IGFBP5 | TLR2 | NOMO1 | TPI1 | HK3 | RIPK2 |
| GSTO1 | PABPC3 | DCSTAMP | MARCO | MMP12 | RNF180 | VAMP2 |
| SPDYE6 | CSPG4 | TNFSF13B | ARL8B | VMO1 | IBSP | CA5B |
| MATN2 | SCARNA27 | GMDS | BLVRB | DARS | MMP8 | OR9A2 |
| OSBPL8 | TGFB2 | CTPS2 | | PABPC3 | IL13 | ZNF267 |
| NOMO3 | SNAPC1 | MKX | | SLC36A4 | LUCAT1 | CEP55 |
| UBE2D1 | TIMP4 | LUCAT1 | | AGTPBP1 | PIK3CB | CYTH3 |
| BMP6 | CCND2 | RAB8B | | HLA_G_3 | CTPS2 | PDCD11 |
| YPEL2 | RAB8B | | | PSD3 | ZNF426 | INSR |
| APOC2 | AOX1 | | | ADAM12 | NHLRC3 | SMARCC2 |
| RIPK2 | RAPGEF4 | | | OSBPL8 | CEP170 | ASS1 |
| TMOD1 | LDB2 | | | SEMA3C | PCDHB12 | TNFSF13B |
| SPDYE2_1 | BBS5 | | | MTUS1 | MNDA | SCARNA27 |
| JAKMIP2 | IVNS1ABP | | | ZFYVE16 | LSP1 | HTRA4 |
| ABCA1 | IL8 | | | SLC2A1 | SGCB | ATF2 |
| GGA2_1 | CD109 | | | NUDCD2 | SNORD116_2_1 | CSF1 |
| ENG | RAMP3 | | | SREBF1 | TRAF5 | BID |
| NHLRC3 | RELB | | | MYL12A | FUCA2 | MMP2 |
| GPX2 | TDO2 | | | CD109 | MARCH1 | MREG |
| EPB41L3 | CCDC88A | | | MIR125B1 | FAM35BP | VWF |
| IGFBP3 | CPM | | | ERH | MIR718 | WDR26 |
| TNFSF13B | GMFB | | | LRP12 | FAM126A | RNFT1 |
| SREBF1 | ADAM8 | | | MLF1IP | HPRT1 | HK2 |
| ARL8B | SLC39A8 | | | LINC00478 | AKT3 | TGFBR3 |
| ARHGAP29 | FMNL2 | | | JAKMIP2 | LYSMD2 | CLDN23 |
| MAML2 | LILRA2 | | | RAPGEF4 | CCND2 | CEP170P1 |
| EPHA4 | CTSB | | | TDO2 | FMN1 | CXCR2P1 |
| MIOS | FAP | | | KLKB1 | CTSD | TLR6 |
| DSC2 | TLR5 | | | P4HA1 | IL8 | RUNX1T1 |
| TGFBR2 | FAM134B | | | CPM | CLEC2B | ZCCHC6 |
| NR4A3 | ARHGAP18 | | | GSTO1 | TMOD3 | IL1RN |
| IRAK3 | STX4 | | | IFNAR1 | UBE2D1 | MYOCD |
| ASS1 | TMEM136 | | | MAML2 | C2CD2 | NABP1 |
| IFNAR1 | FABP5 | | | ITGB8 | DLC1 | CNN1 |
| ZNF350 | KCTD12 | | | EPB41L3 | MIR3153 | |
| ZCCHC6 | | | | ARHGAP11A | KLF8 | |

Table 5 below includes the full performance metrics listing for the set of transcripts robustly estimated on a continuous expression level basis, including the relative weighting of plaque morphology tissue types utilized by the estimation models, illustrating model performance for examples of these transcripts. Examples of transcripts well estimated by morphological assessment combined with clinical variables included transcripts associated with immune regulation, e.g., Cluster of Differentiation 72 (CD72) (CCC=0.4, slope=0.7), 7 Deleted in Liver Cancer 1 (DLC1) (CCC=0.4, slope=0.7), and Intercellular Adhesion Molecule 1 (ICAM1) (CCC=0.3, slope=0.6), 8 acyltransferase activity (ZDHHC6) (CCC=0.4, slope=0.9), 9 and a number of matrix metalloproteinases including MMP1210, 11. High CALC was associated with high expression levels of Proteoglycan 4 (PRG4) and low levels of Speedy/RINGO Cell Cycle Regulator Family Member E1 (SPDYE1), as examples (Table 5, below).

TABLE 5
Performance Metrics for Top Transcripts: Continuous

| response | predictorSet | modelType | RMSE | | Rsquared | | CCC | | bias | |
|---|---|---|---|---|---|---|---|---|---|---|
| MIR125B1 | Morphology + | rfeLR | 0.35 | [0.32, 0.38] | 0.66 | [0.61, 0.7] | 0.51 | [0.48, 0.55] | −0.02 | [−0.05, 0.02] |
| | Clinical + Demog | | | | | | | | | |
| ZDHHC6 | Morphology + | ridge | 0.29 | [0.25, 0.32] | 0.57 | [0.48, 0.67] | 0.43 | [0.38, 0.49] | −0.01 | [−0.06, 0.04] |
| | Clinical + Demog | | | | | | | | | |
| ASS1 | Morphology + | cubist | 0.29 | [0.25, 0.33] | 0.67 | [0.58, 0.75] | 0.5 | [0.42, 0.58] | −0.01 | [−0.06, 0.03] |
| | Clinical + Demog | | | | | | | | | |
| FAM35A | Morphology | avNNet | 7.95 | [0, 2.68] | 0.61 | [0.51, 0.7] | 0.48 | [0.42, 0.54] | 7.77 | [5.25, 10.28] |
| MMP12 | Morphology + | ridge | 2.96 | [2.6, 3.32] | 0.61 | [0.5, 0.71] | 0.42 | [0.34, 0.51] | 0.02 | [−0.44, 0.48] |
| | Clinical + Demog | | | | | | | | | |
| IL1B | Morphology + | ridge | 1.18 | [0.96, 1.39] | 0.56 | [0.45, 0.67] | 0.38 | [0.29, 0.46] | −0.11 | [−0.37, 0.14] |
| | Clinical + Demog | | | | | | | | | |
| DLC1 | Morphology + | rfeLR | 0.54 | [0.51, 0.58] | 0.55 | [0.5, 0.61] | 0.4 | [0.36, 0.44] | 0.03 | [−0.01, 0.08] |
| | Clinical + Demog | | | | | | | | | |
| CD72 | Morphology + | pls | 0.32 | [0.28, 0.35] | 0.64 | [0.56, 0.72] | 0.42 | [0.34, 0.5] | −0.01 | [−0.06, 0.04] |
| | Clinical + Demog | | | | | | | | | |
| SPDYE1 | Morphology | ridge | 0.19 | [0.17, 0.22] | 0.62 | [0.53, 0.72] | 0.44 | [0.38, 0.51] | 0 | [−0.04, 0.04] |
| ARAP1_AS1_yj | Morphology | avNNet | 3.94 | [0, 1.42] | 0.66 | [0.55, 0.76] | 0.49 | [0.43, 0.55] | 3.77 | [2.53, 5.01] |
| NUDT21 | Morphology + | avNNet | 10.38 | [0, 3.46] | 0.58 | [0.48, 0.68] | 0.43 | [0.38, 0.49] | 10.1 | [6.82, 13.38] |
| | Clinical + Demog | | | | | | | | | |
| IGF2BP2 | Morphology + | rfeLR | 0.53 | [0.48, 0.57] | 0.46 | [0.42, 0.51] | 0.35 | [0.31, 0.38] | 0.01 | [−0.03, 0.05] |
| | Clinical + Demog | | | | | | | | | |
| ST6GALNAC6 | Morphology | avNNet | 6.4 | [0, 2.16] | 0.65 | [0.54, 0.75] | 0.48 | [0.41, 0.54] | 6.17 | [4.16, 8.18] |
| ARAP1_AS1 | Morphology | avNNet | 3.94 | [0, 1.41] | 0.64 | [0.55, 0.73] | 0.47 | [0.39, 0.55] | 3.76 | [2.53, 4.99] |
| CDKN2A | Morphology | avNNet | 4.89 | [0, 1.71] | 0.65 | [0.56, 0.73] | 0.47 | [0.42, 0.52] | 4.78 | [3.23, 6.33] |
| SPDYE2 | Morphology + | cubist | 0.27 | [0.23, 0.3] | 0.63 | [0.54, 0.72] | 0.44 | [0.39, 0.49] | 0.02 | [−0.03, 0.07] |
| | Clinical + Demog | | | | | | | | | |
| GATM | Morphology | avNNet | 7.52 | [0, 2.48] | 0.64 | [0.55, 0.72] | 0.46 | [0.4, 0.52] | 6.96 | [4.64, 9.28] |
| ADAM8 | Morphology + | rfeLR | 0.45 | [0.42, 0.48] | 0.53 | [0.48, 0.58] | 0.4 | [0.37, 0.43] | −0.02 | [−0.06, 0.02] |
| | Clinical + Demog | | | | | | | | | |
| SMARCC2 | Morphology + | avNNet | 9.4 | [0, 3.13] | 0.64 | [0.54, 0.73] | 0.43 | [0.36, 0.49] | 9.19 | [6.2, 12.17] |
| | Clinical + Demog | | | | | | | | | |
| MMP8 | Morphology + | rfeLR | 1.35 | [1.25, 1.44] | 0.55 | [0.5, 0.6] | 0.39 | [0.35, 0.43] | 0.03 | [−0.08, 0.14] |
| | Clinical + Demog | | | | | | | | | |
| PRDX6 | Morphology + | avNNet | 8.52 | [0, 2.85] | 0.49 | [0.39, 0.59] | 0.37 | [0.3, 0.44] | 8.21 | [5.53, 10.89] |
| | Clinical + Demog | | | | | | | | | |
| SLC30A1 | Morphology | avNNet | 9.62 | [0, 3.24] | 0.6 | [0.49, 0.71] | 0.4 | [0.31, 0.49] | 9.35 | [6.31, 12.38] |
| GMDS | Morphology + | rfeLR | 0.32 | [0.3, 0.33] | 0.49 | [0.44, 0.53] | 0.39 | [0.36, 0.42] | 0.01 | [−0.02, 0.03] |
| | Clinical + Demog | | | | | | | | | |
| YPEL2_yj | Morphology + | pls | 0.5 | [0.44, 0.57] | 0.47 | [0.36, 0.58] | 0.31 | [0.23, 0.4] | 0.05 | [−0.05, 0.16] |
| | Clinical + Demog | | | | | | | | | |
| YPEL2 | Morphology | avNNet | 8.78 | [0, 2.92] | 0.57 | [0.47, 0.67] | 0.37 | [0.29, 0.46] | 8.44 | [5.69, 11.19] |
| PRG4 | Morphology + | pls | 2.07 | [1.75, 2.38] | 0.6 | [0.49, 0.71] | 0.34 | [0.25, 0.44] | 0.08 | [−0.26, 0.43] |
| | Clinical + Demog | | | | | | | | | |
| IL17B | Morphology + | pls | 0.25 | [0.22, 0.29] | 0.49 | [0.39, 0.59] | −0.24 | [0, −0.15] | −0.01 | [−0.04, 0.03] |
| | Clinical + Demog | | | | | | | | | |
| ICAM1 | Morphology + | rfeLR | 0.78 | [0.74, 0.82] | 0.45 | [0.4, 0.5] | 0.32 | [0.27, 0.36] | −0.06 | [−0.12, −0.01] |
| | Clinical + Demog | | | | | | | | | |
| PSMD14 | Morphology | avNNet | 8.21 | [0, 2.8] | 0.51 | [0.4, 0.62] | 0.35 | [0.26, 0.44] | 7.89 | [5.31, 10.47] |
| MREG | Morphology + | cubist | 0.43 | [0.36, 0.49] | 0.45 | [0.34, 0.56] | 0.35 | [0.27, 0.44] | 0.01 | [−0.05, 0.07] |
| | Clinical + Demog | | | | | | | | | |
| IL4I1_yj | Morphology | avNNet | 5.1 | [0, 1.73] | 0.59 | [0.49, 0.7] | 0.41 | [0.33, 0.48] | 4.65 | [3.09, 6.21] |
| IL17A | Morphology + | rfeLR | 0.18 | [0.17, 0.19] | 0.44 | [0.39, 0.49] | 0.32 | [0.29, 0.36] | 0 | [−0.01, 0.02] |
| | Clinical + Demog | | | | | | | | | |
| APOC2 | Morphology + | ridge | 0.58 | [0.52, 0.64] | 0.46 | [0.35, 0.56] | 0.32 | [0.24, 0.39] | 0 | [−0.12, 0.12] |
| | Clinical + Demog | | | | | | | | | |
| NR4A3 | Morphology | cubist | 1.32 | [1.1, 1.53] | 0.55 | [0.45, 0.64] | 0.37 | [0.31, 0.44] | 0.13 | [−0.13, 0.4] |
| IL4I1 | Morphology + | rfeLR | 0.95 | [0.86, 1.05] | 0.44 | [0.39, 0.49] | 0.28 | [0.25, 0.32] | −0.08 | [−0.16, 0] |
| | Clinical + Demog | | | | | | | | | |
| ZFYVE16 | Morphology + | cubist | 0.71 | [0.62, 0.79] | 0.46 | [0.36, 0.56] | 0.3 | [0.21, 0.4] | 0.04 | [−0.06, 0.15] |
| | Clinical + Demog | | | | | | | | | |
| NR4A3_yj | Morphology | avNNet | 1.11 | [0, 0.54] | 0.56 | [0.45, 0.67] | 0.38 | [0.31, 0.46] | 1.07 | [0.73, 1.42] |
| PRRT3_AS1 | Morphology + | rfeLR | 0.37 | [0.35, 0.4] | 0.56 | [0.5, 0.61] | 0.32 | [0.27, 0.38] | 0.01 | [−0.02, 0.04] |
| | Clinical + Demog | | | | | | | | | |
| SEMA3C | Morphology + | avNNet | 8.63 | [0, 2.89] | 0.5 | [0.4, 0.59] | 0.29 | [0.19, 0.39] | 8.12 | [5.44, 10.81] |
| | Clinical + Demog | | | | | | | | | |
| MIR142 | Morphology | avNNet | 5.11 | [0, 1.76] | 0.5 | [0.39, 0.61] | 0.33 | [0.25, 0.42] | 4.42 | [2.89, 5.95] |
| BLVRB | Morphology | avNNet | 8.01 | [0, 2.59] | 0.43 | [0.33, 0.53] | 0.22 | [0.11, 0.32] | 7.09 | [4.66, 9.53] |
| DCSTAMP | Morphology | avNNet | 5.12 | [0, 1.71] | 0.45 | [0.35, 0.56] | 0.28 | [0.18, 0.38] | 4.33 | [2.82, 5.83] |

| response | slope | | intercept | | CCC_x_slope | CALC | LRNC | IPH | MATX |
|---|---|---|---|---|---|---|---|---|---|
| MIR125B1 | 0.83 | [0.61, 1.05] | 0.81 | [−0.28, 1.91] | 0.43 | 0.79 | | | 0.76 |
| ZDHHC6 | 0.86 | [0.55, 1.17] | 1.34 | [−1.66, 4.33] | 0.37 | 1.00 | 0.49 | | 0.65 |
| ASS1 | 0.74 | [0.56, 0.93] | 1.84 | [0.49, 3.18] | 0.37 | 1.00 | | | 0.50 |
| FAM35A | 0.71 | [0.49, 0.93] | 7.91 | [7.79, 8.03] | 0.34 | 0.83 | 0.45 | | 0.86 |
| MMP12 | 0.8 | [0.53, 1.07] | 2.52 | [−0.93, 5.96] | 0.34 | 0.38 | 0.20 | | 1.00 |
| IL1B | 0.82 | [0.49, 1.15] | 1.22 | [−1.31, 3.76] | 0.31 | 0.22 | 0.30 | | 0.68 |
| DLC1 | 0.74 | [0.44, 1.04] | 2.02 | [−0.24, 4.29] | 0.30 | | | | |
| CD72 | 0.69 | [0.46, 0.91] | 1.68 | [0.48, 2.87] | 0.29 | 0.35 | 0.21 | 0.04 | 0.22 |
| SPDYE1 | 0.64 | [0.38, 0.91] | 2.32 | [0.6, 4.04] | 0.29 | 0.80 | 0.00 | | 1.00 |
| ARAP1_AS1_yj | 0.59 | [0.38, 0.8] | 3.91 | [3.82, 4.01] | 0.29 | 1.00 | 0.29 | 0.48 | 0.31 |
| NUDT21 | 0.63 | [0.39, 0.87] | 10.32 | [10.16, 10.48] | 0.27 | 0.84 | 0.21 | 0.38 | 1.00 |
| IGF2BP2 | 0.79 | [0.38, 1.19] | 1.09 | [−0.95, 3.12] | 0.27 | | | 0.58 | |
| ST6GALNAC6 | 0.56 | [0.38, 0.74] | 6.53 | [6.37, 6.69] | 0.27 | 0.91 | 0.44 | | 1.00 |
| ARAP1_AS1 | 0.57 | [0.38, 0.75] | 3.92 | [3.84, 4] | 0.26 | 1.00 | 0.29 | 0.48 | 0.31 |
| CDKN2A | 0.55 | [0.36, 0.74] | 4.93 | [4.86, 5.01] | 0.26 | 0.69 | 0.21 | | 0.66 |
| SPDYE2 | 0.57 | [0.31, 0.84] | 2.81 | [1.08, 4.54] | 0.25 | 1.00 | | | 0.00 |
| GATM | 0.53 | [0.34, 0.72] | 7.65 | [7.33, 7.97] | 0.24 | 0.46 | 0.70 | | 1.00 |
| ADAM8 | 0.61 | [0.31, 0.9] | 2.43 | [0.58, 4.28] | 0.24 | | | 0.33 | |
| SMARCC2 | 0.57 | [0.32, 0.81] | 9.51 | [9.32, 9.7] | 0.24 | 1.00 | 0.57 | 0.71 | |
| MMP8 | 0.59 | [0.35, 0.83] | 2.46 | [0.99, 3.93] | 0.23 | | | | 0.95 |
| PRDX6 | 0.6 | [0.32, 0.88] | 8.55 | [8.29, 8.8] | 0.22 | 0.89 | 0.21 | 0.47 | 1.00 |
| SLC30A1 | 0.55 | [0.33, 0.77] | 9.6 | [9.45, 9.76] | 0.22 | 0.72 | 0.65 | | 0.96 |
| GMDS | 0.53 | [0.27, 0.78] | 3.68 | [1.71, 5.65] | 0.21 | | | 0.45 | |
| YPEL2_yj | 0.65 | [0.31, 1] | 3.24 | [0.09, 6.39] | 0.20 | 0.35 | 0.30 | | 0.70 |
| YPEL2 | 0.54 | [0.31, 0.77] | 8.79 | [8.58, 8.99] | 0.20 | 0.47 | 0.44 | | 0.65 |
| PRG4 | 0.58 | [0.3, 0.86] | 3.45 | [1.11, 5.79] | 0.20 | 1.00 | 0.00 | | |
| IL17B | −0.79 | [−1.26, −0.31] | 7.25 | [5.31, 9.19] | 0.19 | 0.78 | 0.13 | 0.27 | 0.53 |
| ICAM1 | 0.57 | [0.31, 0.83] | 3.74 | [1.42, 6.07] | 0.18 | 0.52 | | | 0.47 |
| PSMD14 | 0.51 | [0.25, 0.77] | 8.25 | [8.04, 8.46] | 0.18 | 0.26 | 1.00 | 0.19 | |
| MREG | 0.5 | [0.24, 0.77] | 3.02 | [1.41, 4.63] | 0.18 | 1.00 | | 0.00 | 1.00 |
| IL4I1_yj | 0.43 | [0.27, 0.59] | 5.1 | [4.94, 5.25] | 0.18 | 0.70 | 0.90 | 0.04 | 1.00 |
| IL17A | 0.55 | [0.22, 0.88] | 1.53 | [0.41, 2.65] | 0.18 | 0.59 | | | |
| APOC2 | 0.55 | [0.23, 0.88] | 2.52 | [0.69, 4.36] | 0.18 | 0.55 | 0.47 | | |
| NR4A3 | 0.45 | [0.17, 0.72] | 4.01 | [2.05, 5.97] | 0.17 | 0.00 | 0.00 | 0.00 | 0.00 |
| IL4I1 | 0.58 | [0.11, 1.06] | 2.2 | [−0.42, 4.82] | 0.16 | | | | |
| ZFYVE16 | 0.52 | [0.23, 0.8] | 4.75 | [1.97, 7.52] | 0.16 | 0.00 | | | 1.00 |
| NR4A3_yj | 0.38 | [0.19, 0.57] | 1.13 | [1.11, 1.15] | 0.15 | 0.13 | 0.50 | | 0.49 |
| PRRT3_AS1 | 0.45 | [0.17, 0.73] | 4.29 | [2.13, 6.45] | 0.14 | | 0.89 | | |
| SEMA3C | 0.49 | [0.22, 0.76] | 8.75 | [8.39, 9.1] | 0.14 | 0.49 | 0.16 | | 0.78 |
| MIR142 | 0.34 | [0.11, 0.56] | 5.36 | [5.01, 5.71] | 0.11 | 0.35 | 0.54 | 0.28 | 0.61 |
| BLVRB | 0.49 | [0.18, 0.8] | 8.06 | [7.42, 8.69] | 0.11 | 0.05 | 0.42 | | 0.90 |
| DCSTAMP | 0.34 | [0.13, 0.55] | 5.25 | [4.91, 5.58] | 0.10 | 0.58 | 0.59 | 0.37 | 0.83 |

High LRNC, was for example coupled to high expression of Matrix Metalloproteinase 12 (MMP12) and low levels of Rap Guanine Nucleotide Exchange Factor 4 (RAPGEF4). IPH was strongly related to higher expression of Biliverdin Reductase B (BLVRB) and Cyclin-Dependent Kinase Inhibitor 2A (CDKN2A), but lower levels of Nodal Modulator 1 (NOMO1). Matrix (MATX) was more nuanced, likely as it represents less defined tissue types, and was associated with Interleukin-13 (IL13), and lower levels of Nudix Hydrolase 21 (NUDT21). Several other genes were also coupled to particular tissue types by these analyses, both with and without previous associations to atherosclerosis (Table 6, below).

TABLE 6
Performance Metrics for Top Transcripts: Dichotomized

| response | predictorSet | modelType | ROC | | Sens | | Spec | |
|---|---|---|---|---|---|---|---|---|
| IGF2BP2_d | Morphology + Clinical + Demog | avNNet | 1 | | 1 | | 1 | |
| CDKN2A_d | Morphology + Clinical + Demog | avNNet | 1 | | 1 | | 1 | |
| ICAM1_d | Morphology + Clinical + Demog | avNNet | 1 | | 1 | | 1 | |
| APOC2_d | Morphology + Clinical + Demog | avNNet | 1 | | 1 | | 1 | |
| NR4A3_d | Morphology + Clinical + Demog | avNNet | 1 | | 1 | | 1 | |
| NUDT21_d | Morphology + Clinical + Demog | avNNet | 1 | | 1 | | 1 | |
| ADAM8_d | Morphology + Clinical + Demog | avNNet | 1 | | 1 | | 1 | |
| DLC1_d | Morphology | rfeLR | 0.98 | [0.96, 0.99] | 0.9 | [0.86, 0.94] | 0.89 | [0.85, 0.92] |
| MMP8_d | Morphology | c50 | 0.92 | [0.87, 0.96] | 0.89 | [0.82, 0.96] | 0.95 | [0.9, 1] |
| CD72_d | Morphology + Clinical + Demog | rfeLR | 0.91 | [0.89, 0.94] | 0.92 | [0.9, 0.95] | 0.88 | [0.84, 0.91] |
| SPDYE1_d | Morphology | svmRadial | 0.96 | [0.93, 0.99] | 0.87 | [0.8, 0.94] | 0.89 | [0.82, 0.96] |
| ARAP1_AS1_d | Morphology | rfeLR | 0.96 | [0.94, 0.97] | 0.86 | [0.83, 0.9] | 0.88 | [0.84, 0.91] |
| MIR125B1_d | Morphology | avNNet | 0.95 | [0.91, 0.99] | 0.89 | [0.82, 0.96] | 0.82 | [0.73, 0.92] |
| PRRT3_AS1_d | Morphology | svmRadial | 0.93 | [0.87, 0.98] | 0.84 | [0.76, 0.92] | 0.89 | [0.81, 0.96] |
| PRDX6_d | Morphology | avNNet | 0.92 | [0.86, 0.97] | 0.92 | [0.87, 0.98] | 0.8 | [0.71, 0.89] |
| SPDYE2_d | Clinical | c50 | 0.88 | [0.82, 0.93] | 0.91 | [0.85, 0.97] | 0.84 | [0.75, 0.92] |
| ST6GALNAC6_d | Morphology | avNNet | 0.93 | [0.88, 0.98] | 0.86 | [0.79, 0.93] | 0.85 | [0.78, 0.92] |
| ZDHHC6_d | Morphology + Clinical + Demog | rfeLR | 0.94 | [0.92, 0.96] | 0.85 | [0.81, 0.89] | 0.84 | [0.79, 0.88] |
| GMDS_d | Morphology | svmRadial | 0.91 | [0.85, 0.97] | 0.89 | [0.81, 0.96] | 0.81 | [0.72, 0.91] |
| MMP12_d | Morphology | svmRadial | 0.89 | [0.83, 0.94] | 0.82 | [0.75, 0.9] | 0.89 | [0.82, 0.96] |
| PSMD14_d | Morphology | avNNet | 0.88 | [0.82, 0.94] | 0.84 | [0.76, 0.91] | 0.88 | [0.8, 0.95] |
| MIR142_d | Morphology + Clinical + Demog | pls | 0.9 | [0.85, 0.95] | 0.79 | [0.69, 0.88] | 0.89 | [0.82, 0.96] |
| IL4I1_d | Morphology | svmRadial | 0.9 | [0.84, 0.96] | 0.85 | [0.78, 0.92] | 0.82 | [0.74, 0.91] |
| SMARCC2_d | Morphology + Clinical + Demog | rfeLR | 0.88 | [0.85, 0.91] | 0.8 | [0.75, 0.85] | 0.88 | [0.84, 0.91] |
| IL17B_d | Morphology | svmRadial | 0.86 | [0.79, 0.93] | 0.81 | [0.73, 0.9] | 0.84 | [0.75, 0.93] |
| GATM_d | Morphology | svmRadial | 0.87 | [0.81, 0.93] | 0.76 | [0.67, 0.85] | 0.88 | [0.8, 0.95] |
| YPEL2_d | Morphology | svmRadial | 0.88 | [0.81, 0.94] | 0.79 | [0.71, 0.87] | 0.84 | [0.75, 0.92] |
| SEMA3C_d | Morphology | avNNet | 0.86 | [0.78, 0.93] | 0.82 | [0.72, 0.91] | 0.81 | [0.72, 0.91] |
| ASS1_d | Morphology + Clinical + Demog | svmRadial | 0.89 | [0.83, 0.94] | 0.84 | [0.76, 0.91] | 0.75 | [0.65, 0.85] |
| ZFYVE16_d | Morphology | avNNet | 0.87 | [0.8, 0.94] | 0.76 | [0.67, 0.86] | 0.84 | [0.75, 0.93] |
| FAM35A_d | Morphology + Clinical + Demog | avNNet | 1 | | 0.5 | | 1 | |
| PRG4_d | Morphology | avNNet | 0.85 | [0.77, 0.93] | 0.78 | [0.69, 0.86] | 0.75 | [0.65, 0.85] |
| BLVRB_d | Morphology + Clinical + Demog | c50 | 0.78 | [0.71, 0.85] | 0.69 | [0.59, 0.79] | 0.85 | [0.76, 0.94] |
| DCSTAMP_d | Morphology | svmRadial | 0.78 | [0.7, 0.86] | 0.82 | [0.75, 0.9] | 0.69 | [0.59, 0.79] |
| MREG_d | Morphology + Clinical + Demog | svmRadial | 0.81 | [0.71, 0.9] | 0.65 | [0.53, 0.77] | 0.78 | [0.67, 0.88] |
| SLC30A1_d | Morphology | svmRadial | 0.81 | [0.73, 0.89] | 0.69 | [0.57, 0.81] | 0.72 | [0.61, 0.84] |
| IL17A_d | Morphology + Clinical + Demog | pls | 0.74 | [0.65, 0.83] | 0.8 | [0.71, 0.89] | 0.65 | [0.52, 0.78] |
| IL1B_d | Morphology + Clinical + Demog | svmRadial | 0.83 | [0.76, 0.91] | 0.91 | [0.84, 0.98] | 0.48 | [0.37, 0.58] |

| response | Kappa | | AUC_x_Kappa | CALC | LRNC | IPH | MATX |
|---|---|---|---|---|---|---|---|
| IGF2BP2_d | 1 | | 1.00 | 0.12 | 0.41 | | 1.00 |
| CDKN2A_d | 1 | | 1.00 | 0.71 | 0.22 | 0.56 | 1.00 |
| ICAM1_d | 1 | | 1.00 | 0.68 | 1.00 | 0.01 | 0.59 |
| APOC2_d | 1 | | 1.00 | 1.00 | 0.30 | 0.56 | 1.00 |
| NR4A3_d | 1 | | 1.00 | 0.08 | 0.48 | 0.35 | 0.49 |
| NUDT21_d | 1 | | 1.00 | 0.67 | 0.13 | 0.48 | 1.00 |
| ADAM8_d | 1 | | 1.00 | 0.56 | 0.65 | 0.13 | 1.00 |
| DLC1_d | 0.79 | [0.73, 0.84] | 0.77 | 1.00 | 0.40 | | 0.58 |
| MMP8_d | 0.84 | [0.75, 0.92] | 0.77 | 1.00 | 0.00 | 0.00 | 0.68 |
| CD72_d | 0.8 | [0.75, 0.85] | 0.73 | 0.43 | | | 0.42 |
| SPDYE1_d | 0.74 | [0.65, 0.84] | 0.71 | 1.00 | 0.00 | | 0.08 |
| ARAP1_AS1_d | 0.74 | [0.69, 0.79] | 0.71 | | 1.00 | 0.29 | |
| MIR125B1_d | 0.71 | [0.6, 0.83] | 0.68 | 0.38 | 0.10 | 0.17 | 1.00 |
| PRRT3_AS1_d | 0.72 | [0.62, 0.82] | 0.67 | 0.28 | 0.33 | 0.10 | 1.00 |
| PRDX6_d | 0.72 | [0.62, 0.83] | 0.67 | 1.00 | 0.00 | 0.71 | 0.60 |
| SPDYE2_d | 0.75 | [0.65, 0.85] | 0.66 | | | | |
| ST6GALNAC6_d | 0.7 | [0.6, 0.81] | 0.66 | 0.58 | 0.00 | | 1.00 |
| ZDHHC6_d | 0.69 | [0.62, 0.75] | 0.65 | | | | |
| GMDS_d | 0.7 | [0.6, 0.8] | 0.64 | 0.53 | 0.58 | | 1.00 |
| MMP12_d | 0.71 | [0.61, 0.81] | 0.63 | 0.28 | 0.70 | | 1.00 |
| PSMD14_d | 0.71 | [0.61, 0.81] | 0.63 | 0.00 | 0.19 | | 1.00 |
| MIR142_d | 0.68 | [0.57, 0.78] | 0.61 | 0.01 | 0.18 | 0.17 | 0.21 |
| IL4I1_d | 0.68 | [0.57, 0.78] | 0.61 | | | 0.71 | |
| SMARCC2_d | 0.67 | [0.61, 0.73] | 0.59 | | | | |
| IL17B_d | 0.65 | [0.54, 0.76] | 0.56 | 0.50 | | 0.64 | 0.36 |
| GATM_d | 0.64 | [0.54, 0.74] | 0.55 | | | 0.82 | |
| YPEL2_d | 0.62 | [0.52, 0.73] | 0.55 | 1.00 | 0.45 | | 0.56 |
| SEMA3C_d | 0.63 | [0.5, 0.75] | 0.53 | 0.88 | | | 1.00 |
| ASS1_d | 0.59 | [0.47, 0.7] | 0.52 | | 0.51 | 0.49 | 1.00 |
| ZFYVE16_d | 0.6 | [0.48, 0.72] | 0.52 | 1.00 | | | 0.90 |
| FAM35A_d | 0.5 | | 0.50 | 0.70 | 0.08 | | 1.00 |
| PRG4_d | 0.52 | [0.39, 0.66] | 0.45 | 1.00 | 0.21 | | 0.00 |
| BLVRB_d | 0.54 | [0.41, 0.66] | 0.42 | 0.00 | | 0.83 | 0.00 |
| DCSTAMP_d | 0.51 | [0.4, 0.62] | 0.40 | 0.00 | 1.00 | 0.58 | 0.97 |
| MREG_d | 0.42 | [0.26, 0.59] | 0.34 | 0.74 | | 0.21 | 0.88 |
| SLC30A1_d | 0.41 | [0.26, 0.56] | 0.34 | 1.00 | 0.00 | 0.31 | 0.99 |
| IL17A_d | 0.44 | [0.31, 0.57] | 0.33 | 0.97 | 0.01 | | 1.00 |
| IL1B_d | 0.39 | [0.26, 0.52] | 0.32 | 0.46 | | | 1.00 |

In short, in this example, which is not to be considered limiting, 414 transcripts were robustly predicted and coupled to relevant plaque features such as LRNC with biological pathways associated with inflammatory processes and ECM degradation^{49, 50} and IPH with expression of BLVRB and hemoglobin metabolism, as previously reported by our group.³³ In this example, of the 414, 237 met the further criteria for inclusion in pathway analysis as being particularly robustly predicted. Moreover, approximately 100 of the 237 could be estimated more specifically by continuous, actual, value, thus beyond the ability to predict high or low expression only.

While Tables 4, 5, and 6 concretely support the feasibility of the presently described methods, we note that they are mere examples, and that many other transcripts can be identified using the presently described methods. In general, the results in this example demonstrate that transcript expression levels can be estimated from CTA analysis by matching plaque morphology against the expression of transcripts selected based on their relevance to atherosclerotic disease and found in actual tissue samples.

### Example 2: Supervised Models for Estimation of Continuous Gene Expression from Plaque Morphology

We then built models for each transcript to estimate the continuous valued expression level based on plaque morphology.

### Materials and Methods

Statistical Analysis

Supervised model quality (MQ) was determined as the product of two measures for each model type. MQ for continuous estimation models was computed as the product of concordance correlation coefficient (CCC) and regression slope of predicted vs. observed for continuous value estimation (the former to measure the tightness of fit, but augmented by the latter to ensure proportional prediction relative to observed). MQ for dichotomized categoric prediction models was computed as the product of area under the receiver characteristic curve (AUC) times Kappa for dichotomized prediction (the former to measure the net classification performance, but augmented by the latter to ensure performance in both high and low expression classes). Transcripts were classified as “robustly predicted” if dichotomized MQ exceeded 0.15 (e.g. as met by AUC of 0.75 and Kappa 0.2), and were included in unsupervised clustering analyses. Those with MQ exceeding 0.4 (e.g. as met by AUC of 0.8 and Kappa 0.5) were classified as “particularly robustly predicted” and further analysed by gene-set enrichment analysis (GSEA) to elucidate biological processes and molecular pathways at the cohort level, as well as being included in test patient validation. GSEA was conducted using EnrichR (https://amp.pharm.mssm.edu/Enrichr/), further passing results from Gene Ontology Biological process 2018 with p-<0.05 values (adjusted for multi-hypothesis testing) to Revigo (see internet at //revigo.irb.hr/) to determine non-duplicative processes, and finally merged with Reactome 2016 pathways that fell in the same range of significance.

Models were then fixed (“locked down”) and applied to a sequestered set of patients (n=4) selected at random for which ground truth was known, to validate the performance of the model on patients not included in development of the model (“unseen patients”) to test generalizability.⁴⁸ For each test patient, we used the models for transcripts that were particularly robustly predicted and determined the significance of the predictions by applying a bootstrap method to permute plaque morphology inputs to each transcript's model, providing a measure of model stability used to adjust the outputs for each test patient. Model predictions were sorted by individualized confidence and the top 20 most significantly dysregulated transcripts plotted (ranked by combining the degree of dysregulation and the statistical significance in its estimation), for each patient, which was finally compared with the true expression of corresponding transcripts. We then proceeded to pathway analysis by GSEA using the particularly robustly predicted transcripts for each patient to provide a patient-specific unbiased determination of dominant mechanisms. The patient-specific GSEAs were determined from transcript ranking (see above), and p-values for the process were adjusted for multiple hypothesis testing.

Histochemistry and Immunohistochemistry (IHC)

CEA specimens were fixed for 48 hours in 4% Zn-formaldehyde and macro-calcified plaques were de-calcified in Modified Decalcification Solution (HL24150.1000) for 4-6 days at room temperature. Specimens were dehydrated in graded ethanol, embedded in paraffin and sectioned. For histology, slides were deparaffinized and rehydrated in ethanol and stained with Hematoxylin or Masson's Trichrome according to the manufacturer's protocol (Mayers, Sigma-Aldrich, Germany). IPH was detected by Perl's Blue staining (Histolab, Sweden) for 3 min, rinsed and counterstained in nuclear fast red. Slides were finally dehydrated with ethanol and mounted.

All IHC reagents were from Biocare Medical (Concord, Calif.). In brief, 5 μm sections were deparaffinized in Histolab Clear and rehydrated in graded ethanol. For antigen retrieval, slides were subjected to high-pressure boiling in DIVA buffer (pH 6.0). After blocking with Background Sniper, anti-TGFBR2 (Abcam 186838; Cambridge, Mass.), anti-IL1R1 (Abcam 106278) were diluted in Da Vinci Green solution, applied on slides and incubated at room temperature for 1 hour. Isotype rabbit and mouse IgG were used as primary antibodies for negative controls. A probe-polymer system with alkaline phosphatase was applied, with subsequent detection using Warp Red. Slides were counterstained with Hematoxylin QS (Vector Laboratories, Burlingame, Calif.), dehydrated and mounted in Pertex (Histolab, Gothenburg, Sweden). Images were taken in an automated ScanScope slidescanner.

Results

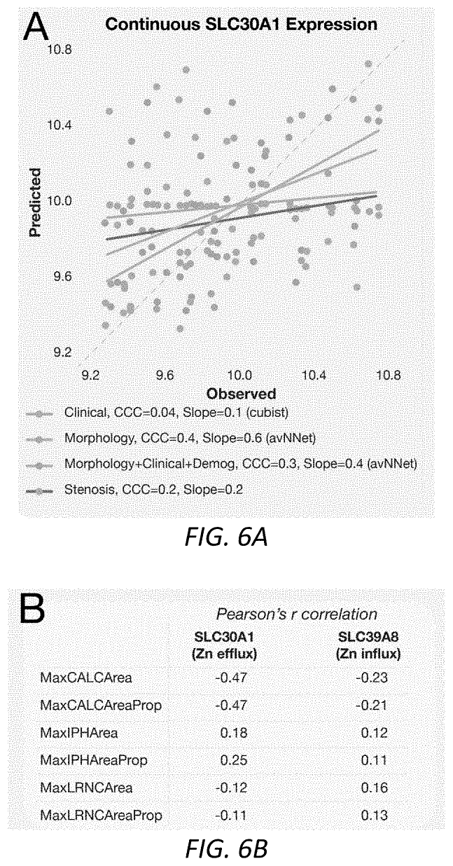

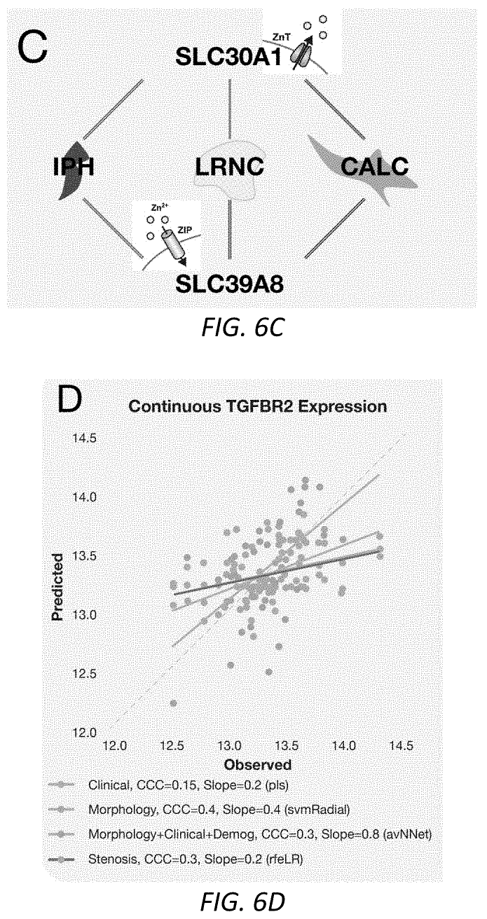

Among those that rated high for model quality, we found transcripts of two functionally different divalent cation transporters where expression clearly associated with plaque morphology, Solute Carrier Family 30 Member 1 (SLC30A1) and Solute Carrier Family 39 Member 8, encoding ZIP8 (FIGS. 6A, 6B). High levels of CALC predicted relatively low, IPH predicted high expression of both transporters, while LRNC predicted high expression of influx (SLCA39A8) but low expression of efflux transporters (SLC30A1; FIGS. 6B, 6C). Specifically, SLC30A1 as an example for which plaque morphology, provided good estimation of gene expression. Continuous-value expression model performance represented by a scatter plot with multiple regression lines where each curve plots the best model fit for each of the different predictor sets considered. SLC30A1 expression prediction performance using plaque morphology was superior to clinical variables (laboratory values), and optimal when used alone rather than in combination (FIG. 6A; points and regression lines in colours annotated at bottom of graph). Table comparing the correlation for each of three tissue types on expression level for two transporter genes representing Zn influx (SLC39A8) and Zn efflux (SLC30A1) (FIG. 6B; Pearson's r correlation shown to indicate relative direction rather than magnitude; MaxCALCArea, MaxIPHArea, and MaxLRNCArea indicates maximum cross-sectional area; Prop suffix indicates proportional occupancy of tissue relative to overall wall area). As IPH increased and CALC decreased, the expression of both transporters increased, but as LRNC increased, expression of influx transporter increased and efflux transporter decreased (FIG. 6C).

Other examples of transcripts for which morphological assessment provided robust continuous-value estimation are listed in Table 5.

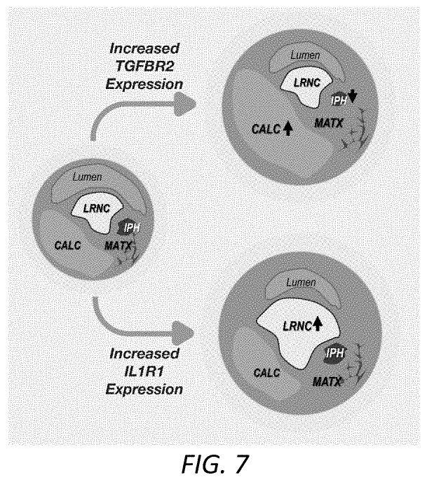

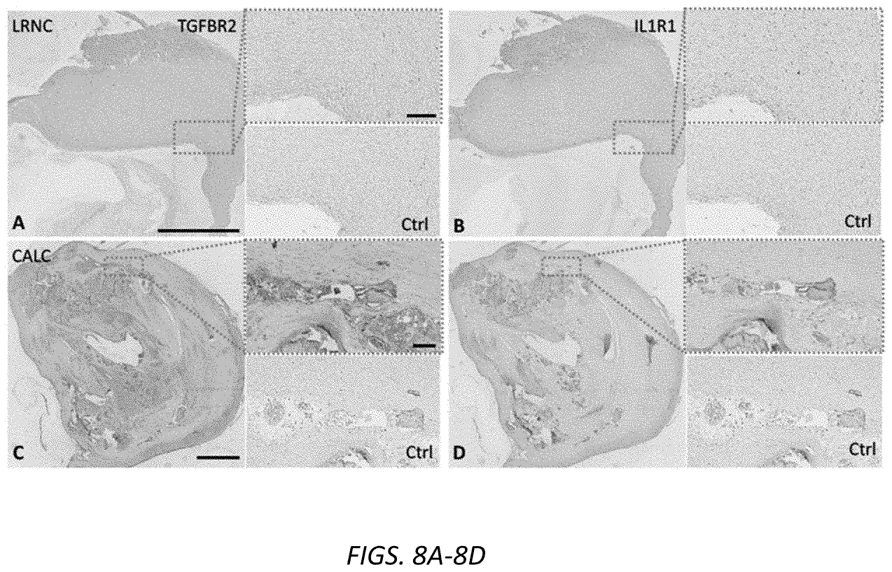

Expression levels of several transcripts could also be assessed through a combination of morphological and clinical variables, which improved determination of continuous-valued expression levels, including a number of cytokines and cytokine receptors. Transforming Growth Factor-beta (TGF-β) receptor type 2 (TGFBR2) was best fit with a model combining plaque morphology and clinical factors (CCC=0.3, slope=0.8; FIG. 6D). Morphological assessment alone was superior to clinical factors alone but improved further when both variables were combined and superior to the degree of stenosis. We also observed an interesting relationship between morphology, TGFBR2 and Interleukin-1 receptor (IL1R1) expression.

Whereas increased levels of TGFBR2 was predicted by lesions with high CALC and with less IPH, plaques with larger LRNC predicted higher IL1R1 expression (FIG. 7). In support of this observation, qualitative immunohistochemical assessment of IL1R1- and TGFBR2-protein expression in CEA specimens demonstrated more IL1R1 staining in plaques with LRNC predominance whereas TGFBR2 was more abundant in lesions with high CALC (FIGS. 8A-8D). Specifically, FIGS. 8A-8D are representative examples of n=21 carotid plaques analysed by immunohistochemistry to demonstrate varying degrees of staining for TGFBR2 (8A and 8C) and IL1R1 (8B and 8D) in plaques dominated by either lipid-rich necrotic core (LRNC) or calcifications (CALC). Note the predominant staining for TGFBR2 in the highly calcified plaque whereas IL1R1-staining was more abundant in the lipid-rich plaque. Protein signal in red. Bars represent 1 mm or 0.1 mm (inserts) and inserts show control staining using isotype-matched primary antibody (Ctrl). Other examples of transcripts well estimated by morphological assessment alone or in combination with clinical variables are provided in Table 5.

As shown, some transcripts demonstrated novel associations between morphological plaque features and expression levels,⁵¹ such as transcripts encoding divalent cation transporters that may mediate effects of nitric oxide (NO),⁵² and contribute to functional regulation of macrophages and SMCs,^{30, 53} whereas others confirmed previously reported relevance in atherosclerotic plaque instability, such as CDKN2A.⁵⁴ Expression levels of several transcripts could also be determined by combining morphological and clinical variables, which improved predictive power and was superior to the degree of stenosis, a clinically used surrogate marker for stroke-risk in patients with carotid stenosis.⁵⁵ For example, in this analysis, IL1R1 expression associated with LRNC and TGFBR2 to highly calcified lesions. Previously, IL1β-mediated immune signalling through IL1R1 has been attributed a key role in atherosclerotic inflammation.⁵⁶ Inhibition of this interaction has been shown to reduce plaque progression in atherosclerotic mice⁵⁷ and improve outcome in patients with CVD.⁵⁸ In contrast, TGFβ and its receptors have been coupled to profibrotic processes and plaque stabilizing effects, which may be consistent with the association of TGBR2 to highly calcified, stable, lesions,³⁷ and here also observed adjacent to macro calcifications by immunohistochemistry. Combination of morphology and clinical variables could also predict expression levels of MMP12, previously reported to be associated with ischemic stroke.⁵⁹

### Example 3: Models for Dichotomized Classification of Gene Expression

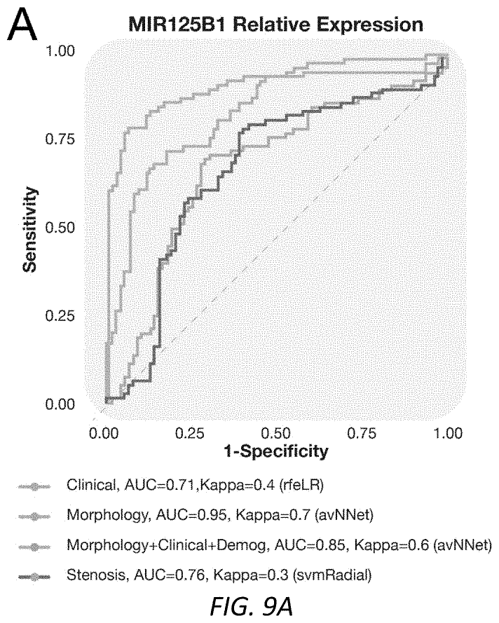

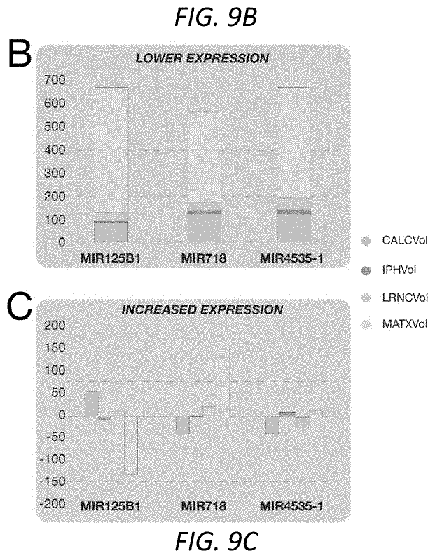

A second set of models were built for dichotomized classification of transcript levels above or below median expression value. MIR125B1, MIR718, and MIR4536-1 were examples where dichotomized expression level (defined as being higher or lower than the median) demonstrated robust classification accuracy. FIG. 9 shows receiver operating characteristic (ROC) curves for classifying MIR125B1 by various predictor sets, where classification performance differed based on the predictors sets used. MIR125B1 expression level was robustly estimated using plaque morphology, superior to clinical variables (laboratory values) and was optimal when used alone rather than in combination (FIG. 9A; ROC curves in colours annotated at bottom of graph). Volumes for plaque tissue components typifying lower (below median) expression of each of the microRNAs demonstrated different distributions of the interrogated tissue types (FIG. 9B). Changes in the plaque composition as expression increased showed even greater differences (FIG. 9C), e.g. higher CALC predict lower expression of MIR125B1 but decreased expression of MIR718 and MIR4535-1. Vertical axes in B and C relative rather than absolute scaling, and colour key is provided at right.

Dichotomized expression (higher vs. lower) of microRNA 125b-1 (MIR125B1) was well classified by morphology (FIG. 9A), as well as MIR718 and MIR4536-1. Lower expression of MIR125B1 occurred in plaques with a combined burden of LRNC and IPH comparable to CALC, whereas expression was higher in smaller plaques with proportionally more CALC (FIG. 9B). In contrast, lower expression of MIR718 was found in smaller plaques with relatively high CALC burden but increased in larger plaques as CALC proportion decreased. Expression of MIR4536-1 was lower in larger plaques with increased CALC burden and decreased further in plaques with less CALC (FIG. 9C). Additional transcripts predicted as relatively high vs. low levels using morphological assessment are listed in Table 4.

In support of the clinical and biological relevance of these findings, pathway analysis of transcripts where expression levels were determined in at least dichotomized form, revealed associations to established biological processes in atherogenesis and plaque instability.

### Example 4: Estimation of Biological Processes Predicted by Plaque Morphology

The biological relevance of transcripts with expression levels predicted in dichotomized form was investigated by gene-set enrichment analysis (GSEA) to expose biological processes elucidated by plaque morphology. Of the 414 robustly predicted transcripts, 237 transcripts were classified as particularly robustly predicted and hence eligible for pathway analysis as evidenced by ranking according to the product of point estimates against an objectively determined cut-off (Table 7, below). Several fundamental processes related to the pathophysiology of atherosclerosis and plaque instability were found to be enriched such as SMC proliferation; ECM organization; collagen degradation; apoptosis, phospholipid and cholesterol efflux; regulation of epithelial to mesenchymal transition, and neutrophil mediated immunity (FIG. 10; showing pathway analysis of the 237 transcripts meeting model quality to be described as particularly robustly predicted (for which both AUC and Kappa were high) at the cohort level as means to identify biological processes significantly determined by the robustly predicted transcripts. Identified biological processes classified by basic atherosclerotic disease mechanisms (colour codes as per the Key).

TABLE 7
237 Transcripts Comprising the Resulting Quantitative Imaging Biomarker Panel

| | Model | | | | | AUC × |
|---|---|---|---|---|---|---|
| Transcript | Type | ROC | Sens | Spec | Kappa | Kappa |
| NR4A3 | rfeLR | 0.96 | 0.88 | 0.95 | 0.82 | 0.791 |
| | | [0.94, 0.98] | [0.84, 0.91] | [0.93, 0.97] | [0.78, 0.87] | |
| DLC1 | rfeLR | 0.93 | 0.89 | 0.9 | 0.79 | 0.733 |
| | | [0.9, 0.96] | [0.85, 0.92] | [0.86, 0.94] | [0.73, 0.85] | |
| SPDYE1 | rfeLR | 0.96 | 0.85 | 0.89 | 0.74 | 0.712 |
| | | [0.95, 0.98] | [0.81, 0.9] | [0.84, 0.93] | [0.68, 0.8] | |
| ARAP1_AS1 | rfeLR | 0.96 | 0.86 | 0.88 | 0.74 | 0.708 |
| | | [0.94, 0.98] | [0.83, 0.9] | [0.83, 0.92] | [0.68, 0.79] | |
| PRRT3_AS1 | svmRadial | 0.92 | 0.88 | 0.88 | 0.75 | 0.693 |
| | | [0.87, 0.97] | [0.81, 0.95] | [0.8, 0.95] | [0.66, 0.85] | |
| ICAM1 | avNNet | 0.91 | 0.92 | 0.84 | 0.76 | 0.691 |
| | | [0.85, 0.97] | [0.87, 0.98] | [0.75, 0.92] | [0.67, 0.86] | |
| MMP8 | c50 | 0.88 | 0.9 | 0.89 | 0.79 | 0.689 |
| | | [0.81, 0.94] | [0.84, 0.96] | [0.81, 0.96] | [0.69, 0.89] | |
| IGF2BP2 | avNNet | 0.91 | 0.9 | 0.85 | 0.75 | 0.684 |
| | | [0.85, 0.97] | [0.84, 0.96] | [0.77, 0.93] | [0.64, 0.86] | |
| MIR142 | svmRadial | 0.94 | 0.91 | 0.81 | 0.72 | 0.684 |
| | | [0.91, 0.98] | [0.85, 0.97] | [0.73, 0.89] | [0.64, 0.81] | |
| MIR125B1 | rfeLR | 0.92 | 0.89 | 0.85 | 0.74 | 0.682 |
| | | [0.9, 0.95] | [0.85, 0.92] | [0.81, 0.89] | [0.69, 0.79] | |
| NUDT21 | avNNet | 0.94 | 0.86 | 0.86 | 0.72 | 0.680 |
| | | [0.9, 0.98] | [0.79, 0.93] | [0.79, 0.93] | [0.63, 0.82] | |
| ST6GALNAC6 | c50 | 0.94 | 0.88 | 0.84 | 0.71 | 0.670 |
| | | [0.9, 0.99] | [0.8, 0.96] | [0.76, 0.91] | [0.6, 0.82] | |
| PRDX6 | avNNet | 0.91 | 0.9 | 0.84 | 0.74 | 0.668 |
| | | [0.84, 0.97] | [0.83, 0.97] | [0.75, 0.92] | [0.63, 0.85] | |
| IL4I1 | svmRadial | 0.91 | 0.85 | 0.89 | 0.74 | 0.668 |
| | | [0.85, 0.96] | [0.78, 0.92] | [0.82, 0.96] | [0.65, 0.83] | |
| ADAM8 | avNNet | 0.92 | 0.89 | 0.82 | 0.71 | 0.659 |
| | | [0.87, 0.98] | [0.82, 0.96] | [0.74, 0.91] | [0.61, 0.81] | |
| PSMD14 | avNNet | 0.89 | 0.84 | 0.9 | 0.74 | 0.655 |
| | | [0.83, 0.95] | [0.76, 0.91] | [0.83, 0.97] | [0.62, 0.85] | |
| APOC2 | avNNet | 0.96 | 0.82 | 0.85 | 0.68 | 0.645 |
| | | [0.92, 1] | [0.75, 0.9] | [0.77, 0.93] | [0.58, 0.77] | |
| ZFYVE16 | svmRadial | 0.87 | 0.88 | 0.86 | 0.74 | 0.641 |
| | | [0.8, 0.94] | [0.8, 0.95] | [0.79, 0.93] | [0.63, 0.85] | |
| ZDHHC6 | svmRadial | 0.93 | 0.81 | 0.88 | 0.69 | 0.640 |
| | | [0.88, 0.98] | [0.73, 0.89] | [0.8, 0.95] | [0.6, 0.77] | |
| SPDYE2 | glmnet | 0.91 | 0.8 | 0.9 | 0.7 | 0.639 |
| | | [0.87, 0.96] | [0.71, 0.89] | [0.84, 0.96] | [0.59, 0.81] | |
| DRAM1 | svmRadial | 0.92 | 0.85 | 0.84 | 0.69 | 0.636 |
| | | [0.88, 0.97] | [0.78, 0.92] | [0.75, 0.93] | [0.58, 0.79] | |
| ZNF350 | svmRadial | 0.94 | 0.82 | 0.85 | 0.68 | 0.633 |
| | | [0.89, 0.98] | [0.74, 0.91] | [0.77, 0.93] | [0.56, 0.79] | |
| LDB2 | rfeLR | 0.9 | 0.92 | 0.78 | 0.7 | 0.632 |
| | | [0.87, 0.93] | [0.89, 0.96] | [0.73, 0.82] | [0.64, 0.76] | |
| EGLN3 | avNNet | 0.87 | 0.85 | 0.88 | 0.72 | 0.628 |
| | | [0.8, 0.93] | [0.78, 0.92] | [0.8, 0.95] | [0.62, 0.83] | |
| LYSMD2 | avNNet | 0.91 | 0.76 | 0.92 | 0.69 | 0.627 |
| | | [0.86, 0.96] | [0.67, 0.85] | [0.86, 0.99] | [0.57, 0.81] | |
| CR1 | avNNet | 0.88 | 0.88 | 0.84 | 0.71 | 0.623 |
| | | [0.82, 0.93] | [0.8, 0.95] | [0.76, 0.91] | [0.62, 0.81] | |
| TMOD1 | avNNet | 0.92 | 0.85 | 0.82 | 0.68 | 0.620 |
| | | [0.87, 0.97] | [0.77, 0.93] | [0.73, 0.92] | [0.56, 0.79] | |
| GCA | svmRadial | 0.92 | 0.8 | 0.88 | 0.68 | 0.620 |
| | | [0.86, 0.98] | [0.71, 0.89] | [0.8, 0.95] | [0.56, 0.79] | |
| GCA | svmRadial | 0.92 | 0.8 | 0.88 | 0.68 | 0.620 |
| | | [0.86, 0.98] | [0.71, 0.89] | [0.8, 0.95] | [0.56, 0.79] | |
| LRP12 | svmRadial | 0.9 | 0.85 | 0.84 | 0.69 | 0.619 |
| | | [0.84, 0.96] | [0.77, 0.93] | [0.75, 0.92] | [0.57, 0.8] | |
| SASH1 | rfeLR | 0.91 | 0.88 | 0.8 | 0.68 | 0.616 |
| | | [0.89, 0.94] | [0.84, 0.91] | [0.76, 0.84] | [0.63, 0.72] | |
| ARHGAP11B | glmnet | 0.86 | 0.85 | 0.86 | 0.71 | 0.615 |
| | | [0.79, 0.94] | [0.76, 0.94] | [0.78, 0.94] | [0.59, 0.83] | |
| SLC36A1 | avNNet | 0.92 | 0.89 | 0.78 | 0.66 | 0.613 |
| | | [0.88, 0.97] | [0.81, 0.96] | [0.69, 0.86] | [0.55, 0.78] | |
| TPI1 | avNNet | 0.84 | 0.89 | 0.84 | 0.72 | 0.612 |
| | | [0.76, 0.93] | [0.82, 0.96] | [0.75, 0.93] | [0.59, 0.86] | |
| MMP12 | glmnet | 0.89 | 0.81 | 0.88 | 0.69 | 0.610 |
| | | [0.83, 0.94] | [0.73, 0.9] | [0.8, 0.95] | [0.58, 0.79] | |
| VMO1 | avNNet | 0.84 | 0.82 | 0.9 | 0.72 | 0.609 |
| | | [0.77, 0.91] | [0.73, 0.92] | [0.84, 0.96] | [0.61, 0.84] | |
| RRS1 | rfeLR | 0.9 | 0.85 | 0.82 | 0.68 | 0.608 |
| | | [0.87, 0.93] | [0.81, 0.89] | [0.78, 0.87] | [0.61, 0.74] | |
| CD109 | glmnet | 0.88 | 0.79 | 0.9 | 0.69 | 0.602 |
| | | [0.81, 0.94] | [0.69, 0.88] | [0.84, 0.96] | [0.55, 0.83] | |
| MMP7 | c50 | 0.9 | 0.86 | 0.8 | 0.66 | 0.598 |
| | | [0.85, 0.95] | [0.78, 0.94] | [0.71, 0.89] | [0.55, 0.77] | |
| HNRNPU_AS1 | svmRadial | 0.94 | 0.78 | 0.86 | 0.64 | 0.598 |
| | | [0.9, 0.98] | [0.67, 0.88] | [0.78, 0.94] | [0.51, 0.76] | |
| DARS | avNNet | 0.87 | 0.85 | 0.84 | 0.69 | 0.595 |
| | | [0.8, 0.93] | [0.78, 0.92] | [0.75, 0.92] | [0.56, 0.81] | |
| PKHD1L1 | avNNet | 0.85 | 0.91 | 0.79 | 0.7 | 0.595 |
| | | [0.79, 0.91] | [0.85, 0.97] | [0.7, 0.88] | [0.58, 0.82] | |
| PABPC3 | avNNet | 0.88 | 0.85 | 0.82 | 0.68 | 0.595 |
| | | [0.81, 0.95] | [0.77, 0.93] | [0.75, 0.9] | [0.56, 0.79] | |
| NHLRC3 | svmRadial | 0.89 | 0.79 | 0.88 | 0.66 | 0.592 |
| | | [0.84, 0.95] | [0.69, 0.88] | [0.8, 0.95] | [0.54, 0.78] | |
| SLC36A4 | glmnet | 0.88 | 0.81 | 0.86 | 0.68 | 0.591 |
| | | [0.81, 0.94] | [0.72, 0.91] | [0.78, 0.94] | [0.56, 0.79] | |
| AGTPBP1 | c50 | 0.88 | 0.81 | 0.86 | 0.67 | 0.589 |
| | | [0.82, 0.94] | [0.73, 0.89] | [0.78, 0.94] | [0.56, 0.78] | |
| ACTG1 | svmRadial | 0.91 | 0.79 | 0.86 | 0.65 | 0.589 |
| | | [0.85, 0.96] | [0.69, 0.89] | [0.78, 0.94] | [0.54, 0.76] | |
| HLA_G_3 | avNNet | 0.87 | 0.8 | 0.88 | 0.68 | 0.589 |
| | | [0.8, 0.94] | [0.7, 0.9] | [0.8, 0.95] | [0.53, 0.82] | |
| ZNF767 | avNNet | 0.94 | 0.8 | 0.84 | 0.62 | 0.586 |
| | | [0.89, 0.98] | [0.71, 0.88] | [0.75, 0.93] | [0.5, 0.75] | |
| OR9A2 | c50 | 0.88 | 0.85 | 0.81 | 0.66 | 0.580 |
| | | [0.82, 0.93] | [0.77, 0.94] | [0.72, 0.91] | [0.54, 0.79] | |
| CEP170P1 | rfeLR | 0.88 | 0.82 | 0.84 | 0.66 | 0.580 |
| | | [0.84, 0.91] | [0.79, 0.86] | [0.8, 0.88] | [0.61, 0.72] | |
| MAML2 | svmRadial | 0.9 | 0.81 | 0.84 | 0.64 | 0.579 |
| | | [0.85, 0.95] | [0.72, 0.89] | [0.75, 0.92] | [0.54, 0.75] | |
| MAML2 | svmRadial | 0.9 | 0.81 | 0.84 | 0.64 | 0.579 |
| | | [0.85, 0.95] | [0.72, 0.89] | [0.75, 0.92] | [0.54, 0.75] | |
| PTPRB | avNNet | 0.87 | 0.88 | 0.79 | 0.66 | 0.576 |
| | | [0.8, 0.94] | [0.8, 0.95] | [0.71, 0.87] | [0.54, 0.78] | |
| CDH5 | rfeLR | 0.87 | 0.86 | 0.8 | 0.66 | 0.576 |
| | | [0.83, 0.9] | [0.83, 0.9] | [0.76, 0.84] | [0.6, 0.72] | |
| GSTO1 | svmRadial | 0.87 | 0.8 | 0.86 | 0.66 | 0.576 |
| | | [0.81, 0.93] | [0.71, 0.89] | [0.78, 0.94] | [0.55, 0.78] | |
| SCARNA27 | svmRadial | 0.91 | 0.8 | 0.82 | 0.62 | 0.570 |
| | | [0.86, 0.97] | [0.71, 0.89] | [0.74, 0.91] | [0.53, 0.72] | |
| PSD3 | avNNet | 0.91 | 0.79 | 0.84 | 0.62 | 0.566 |
| | | [0.84, 0.97] | [0.69, 0.88] | [0.75, 0.92] | [0.52, 0.73] | |
| ADAM12 | svmRadial | 0.89 | 0.91 | 0.72 | 0.64 | 0.566 |
| | | [0.82, 0.96] | [0.85, 0.97] | [0.63, 0.82] | [0.53, 0.75] | |
| OSBPL8 | svmRadial | 0.84 | 0.86 | 0.81 | 0.68 | 0.565 |
| | | [0.77, 0.91] | [0.79, 0.93] | [0.73, 0.89] | [0.55, 0.8] | |
| SPDYE6 | rfeLR | 0.9 | 0.79 | 0.84 | 0.62 | 0.563 |
| | | [0.87, 0.93] | [0.74, 0.83] | [0.8, 0.88] | [0.57, 0.68] | |
| SEMA3C | rfeLR | 0.88 | 0.88 | 0.76 | 0.64 | 0.560 |
| | | [0.85, 0.91] | [0.85, 0.91] | [0.71, 0.81] | [0.58, 0.69] | |
| MTUS1 | avNNet | 0.93 | 0.81 | 0.8 | 0.6 | 0.559 |
| | | [0.89, 0.97] | [0.72, 0.89] | [0.7, 0.9] | [0.48, 0.72] | |
| RAPGEF4 | avNNet | 0.89 | 0.81 | 0.81 | 0.62 | 0.555 |
| | | [0.83, 0.95] | [0.73, 0.9] | [0.73, 0.9] | [0.49, 0.76] | |
| SLC2A1 | avNNet | 0.83 | 0.85 | 0.81 | 0.66 | 0.553 |
| | | [0.76, 0.91] | [0.76, 0.94] | [0.73, 0.9] | [0.54, 0.79] | |
| NUDCD2 | glmnet | 0.9 | 0.84 | 0.78 | 0.61 | 0.552 |
| | | [0.84, 0.96] | [0.76, 0.92] | [0.67, 0.88] | [0.49, 0.74] | |
| MATN2 | svmRadial | 0.9 | 0.79 | 0.82 | 0.61 | 0.551 |
| | | [0.84, 0.96] | [0.69, 0.89] | [0.75, 0.9] | [0.48, 0.74] | |
| FAP | rfeLR | 0.92 | 0.81 | 0.79 | 0.6 | 0.551 |
| | | [0.9, 0.94] | [0.76, 0.86] | [0.75, 0.83] | [0.55, 0.65] | |
| DDX24 | glmnet | 0.88 | 0.79 | 0.84 | 0.62 | 0.551 |
| | | [0.82, 0.94] | [0.69, 0.88] | [0.75, 0.92] | [0.52, 0.73] | |
| GATM | svmRadial | 0.88 | 0.81 | 0.81 | 0.62 | 0.551 |
| | | [0.82, 0.95] | [0.73, 0.9] | [0.73, 0.9] | [0.51, 0.74] | |
| NOMO3 | avNNet | 0.88 | 0.91 | 0.71 | 0.62 | 0.549 |
| | | [0.81, 0.94] | [0.85, 0.97] | [0.6, 0.82] | [0.5, 0.75] | |
| SREBF1 | avNNet | 0.84 | 0.79 | 0.86 | 0.65 | 0.548 |
| | | [0.76, 0.93] | [0.69, 0.89] | [0.79, 0.93] | [0.51, 0.79] | |
| IGFBP6 | c50 | 0.82 | 0.89 | 0.78 | 0.66 | 0.548 |
| | | [0.75, 0.9] | [0.82, 0.96] | [0.67, 0.88] | [0.52, 0.81] | |
| MYL12A | c50 | 0.84 | 0.95 | 0.7 | 0.65 | 0.544 |
| | | [0.78, 0.89] | [0.9, 1] | [0.61, 0.79] | [0.54, 0.76] | |
| CTSD | rfeLR | 0.87 | 0.78 | 0.85 | 0.62 | 0.543 |
| | | [0.84, 0.9] | [0.74, 0.81] | [0.81, 0.89] | [0.56, 0.69] | |
| LINC00685 | avNNet | 0.84 | 0.87 | 0.78 | 0.64 | 0.541 |
| | | [0.76, 0.93] | [0.8, 0.94] | [0.67, 0.88] | [0.52, 0.76] | |
| GABRE | rfeLR | 0.86 | 0.84 | 0.79 | 0.62 | 0.539 |
| | | [0.83, 0.9] | [0.79, 0.88] | [0.74, 0.83] | [0.56, 0.69] | |
| UBE2D1 | svmRadial | 0.88 | 0.78 | 0.84 | 0.61 | 0.536 |
| | | [0.81, 0.94] | [0.69, 0.86] | [0.75, 0.92] | [0.5, 0.72] | |
| KCNJ5 | rfeLR | 0.91 | 0.78 | 0.81 | 0.59 | 0.532 |
| | | [0.87, 0.94] | [0.71, 0.84] | [0.77, 0.86] | [0.52, 0.66] | |
| BMP6 | rfeLR | 0.87 | 0.84 | 0.78 | 0.61 | 0.532 |
| | | [0.83, 0.9] | [0.79, 0.89] | [0.72, 0.83] | [0.54, 0.68] | |
| ACAP2_IT1 | rfeLR | 0.86 | 0.82 | 0.81 | 0.61 | 0.529 |
| | | [0.83, 0.9] | [0.78, 0.86] | [0.76, 0.86] | [0.55, 0.68] | |
| KREMEN1 | avNNet | 0.88 | 0.81 | 0.79 | 0.6 | 0.529 |
| | | [0.82, 0.94] | [0.73, 0.9] | [0.69, 0.88] | [0.48, 0.72] | |
| YPEL2 | svmRadial | 0.84 | 0.8 | 0.82 | 0.62 | 0.527 |
| | | [0.77, 0.92] | [0.72, 0.88] | [0.73, 0.92] | [0.51, 0.74] | |
| ERH | svmRadial | 0.85 | 0.87 | 0.75 | 0.62 | 0.526 |
| | | [0.78, 0.93] | [0.8, 0.94] | [0.65, 0.85] | [0.48, 0.75] | |
| CPM | avNNet | 0.86 | 0.75 | 0.86 | 0.61 | 0.524 |
| | | [0.79, 0.93] | [0.65, 0.85] | [0.79, 0.93] | [0.5, 0.73] | |
| ATG5 | avNNet | 0.89 | 0.75 | 0.84 | 0.59 | 0.521 |
| | | [0.82, 0.96] | [0.66, 0.84] | [0.75, 0.93] | [0.45, 0.73] | |
| FCGR2B | rfeLR | 0.9 | 0.8 | 0.78 | 0.57 | 0.518 |
| | | [0.87, 0.93] | [0.76, 0.84] | [0.72, 0.83] | [0.52, 0.63] | |
| MLF1IP | svmRadial | 0.86 | 0.81 | 0.79 | 0.6 | 0.518 |
| | | [0.78, 0.94] | [0.73, 0.9] | [0.69, 0.88] | [0.48, 0.72] | |
| CADM1 | rfeLR | 0.86 | 0.82 | 0.78 | 0.6 | 0.518 |
| | | [0.83, 0.89] | [0.79, 0.86] | [0.73, 0.82] | [0.55, 0.65] | |
| LINC00478 | svmRadial | 0.88 | 0.85 | 0.74 | 0.59 | 0.514 |
| | | [0.82, 0.93] | [0.78, 0.92] | [0.65, 0.83] | [0.49, 0.69] | |
| HK2 | rfeLR | 0.89 | 0.79 | 0.79 | 0.57 | 0.513 |
| | | [0.87, 0.92] | [0.74, 0.83] | [0.74, 0.83] | [0.52, 0.63] | |
| RIPK2 | c50 | 0.85 | 0.82 | 0.78 | 0.6 | 0.512 |
| | | [0.79, 0.91] | [0.73, 0.92] | [0.69, 0.86] | [0.47, 0.73] | |
| SPDYE2_1 | svmRadial | 0.85 | 0.75 | 0.85 | 0.6 | 0.510 |
| | | [0.77, 0.93] | [0.65, 0.85] | [0.77, 0.93] | [0.49, 0.71] | |
| SLCO2A1 | glmnet | 0.85 | 0.81 | 0.79 | 0.6 | 0.510 |
| | | [0.78, 0.92] | [0.73, 0.89] | [0.71, 0.87] | [0.48, 0.72] | |
| CLDN1 | rfeLR | 0.86 | 0.8 | 0.79 | 0.59 | 0.507 |
| | | [0.83, 0.89] | [0.75, 0.85] | [0.74, 0.84] | [0.52, 0.66] | |
| STXBP1 | rfeLR | 0.86 | 0.8 | 0.79 | 0.59 | 0.507 |
| | | [0.83, 0.9] | [0.75, 0.85] | [0.74, 0.83] | [0.52, 0.65] | |
| JAKMIP2 | c50 | 0.86 | 0.79 | 0.8 | 0.59 | 0.507 |
| | | [0.8, 0.93] | [0.71, 0.87] | [0.72, 0.88] | [0.47, 0.71] | |
| TDO2 | svmRadial | 0.88 | 0.8 | 0.78 | 0.57 | 0.503 |
| | | [0.81, 0.94] | [0.71, 0.89] | [0.68, 0.87] | [0.45, 0.7] | |
| KLKB1 | avNNet | 0.86 | 0.8 | 0.79 | 0.59 | 0.503 |
| | | [0.79, 0.92] | [0.71, 0.89] | [0.69, 0.89] | [0.46, 0.72] | |
| P4HA1 | avNNet | 0.82 | 0.76 | 0.85 | 0.61 | 0.501 |
| | | [0.74, 0.9] | [0.65, 0.88] | [0.78, 0.92] | [0.47, 0.75] | |
| VAMP2 | glmnet | 0.82 | 0.82 | 0.79 | 0.61 | 0.501 |
| | | [0.74, 0.9] | [0.73, 0.92] | [0.7, 0.88] | [0.47, 0.76] | |
| ABCA1 | avNNet | 0.84 | 0.76 | 0.84 | 0.59 | 0.501 |
| | | [0.77, 0.92] | [0.66, 0.86] | [0.75, 0.93] | [0.46, 0.73] | |
| SNORA38_1 | svmRadial | 0.87 | 0.8 | 0.78 | 0.57 | 0.500 |
| | | [0.81, 0.93] | [0.7, 0.9] | [0.67, 0.88] | [0.45, 0.7] | |
| TLR7 | avNNet | 0.85 | 0.78 | 0.81 | 0.59 | 0.499 |
| | | [0.77, 0.93] | [0.68, 0.87] | [0.72, 0.91] | [0.44, 0.73] | |
| IFNAR1 | avNNet | 0.85 | 0.7 | 0.89 | 0.59 | 0.499 |
| | | [0.79, 0.91] | [0.6, 0.8] | [0.82, 0.96] | [0.48, 0.7] | |
| IL3RA | avNNet | 0.85 | 0.78 | 0.81 | 0.58 | 0.499 |
| | | [0.79, 0.92] | [0.7, 0.85] | [0.72, 0.91] | [0.44, 0.72] | |
| GGA2_1 | svmRadial | 0.86 | 0.88 | 0.7 | 0.58 | 0.496 |
| | | [0.79, 0.93] | [0.81, 0.95] | [0.59, 0.81] | [0.45, 0.7] | |
| ENG | svmRadial | 0.86 | 0.82 | 0.76 | 0.58 | 0.496 |
| | | [0.78, 0.93] | [0.74, 0.9] | [0.66, 0.86] | [0.45, 0.71] | |
| VPS26A | rfeLR | 0.84 | 0.8 | 0.79 | 0.59 | 0.496 |
| | | [0.81, 0.88] | [0.75, 0.85] | [0.74, 0.83] | [0.53, 0.65] | |
| TIMP4 | glmnet | 0.84 | 0.8 | 0.79 | 0.59 | 0.496 |
| | | [0.77, 0.91] | [0.71, 0.89] | [0.69, 0.89] | [0.44, 0.73] | |
| LINC00685_1 | avNNet | 0.88 | 0.82 | 0.75 | 0.56 | 0.496 |
| | | [0.82, 0.94] | [0.73, 0.91] | [0.65, 0.85] | [0.44, 0.69] | |
| ITGB8 | glmnet | 0.9 | 0.75 | 0.8 | 0.55 | 0.495 |
| | | [0.84, 0.96] | [0.66, 0.85] | [0.71, 0.89] | [0.43, 0.68] | |
| GPX2 | svmRadial | 0.9 | 0.72 | 0.82 | 0.55 | 0.495 |
| | | [0.84, 0.96] | [0.62, 0.83] | [0.74, 0.91] | [0.44, 0.66] | |
| TTC39B | rfeLR | 0.82 | 0.82 | 0.78 | 0.6 | 0.495 |
| | | [0.78, 0.87] | [0.78, 0.87] | [0.73, 0.82] | [0.52, 0.68] | |
| PGBD5 | rfeLR | 0.86 | 0.76 | 0.81 | 0.57 | 0.494 |
| | | [0.83, 0.89] | [0.71, 0.81] | [0.77, 0.86] | [0.5, 0.65] | |
| EPB41L3 | c50 | 0.83 | 0.8 | 0.79 | 0.59 | 0.490 |
| | | [0.76, 0.91] | [0.72, 0.88] | [0.69, 0.88] | [0.46, 0.71] | |
| ARHGAP11A | rfeLR | 0.87 | 0.8 | 0.76 | 0.56 | 0.489 |
| | | [0.84, 0.9] | [0.75, 0.85] | [0.72, 0.81] | [0.5, 0.62] | |
| IGFBP3 | avNNet | 0.89 | 0.76 | 0.79 | 0.55 | 0.488 |
| | | [0.82, 0.95] | [0.66, 0.86] | [0.69, 0.88] | [0.42, 0.68] | |
| TNFSF13B | svmRadial | 0.86 | 0.76 | 0.8 | 0.56 | 0.485 |
| | | [0.79, 0.93] | [0.66, 0.86] | [0.71, 0.89] | [0.45, 0.68] | |
| ARL8B | avNNet | 0.82 | 0.85 | 0.74 | 0.59 | 0.485 |
| | | [0.74, 0.91] | [0.77, 0.93] | [0.64, 0.84] | [0.46, 0.72] | |
| GTF3A | svmRadial | 0.81 | 0.81 | 0.79 | 0.6 | 0.484 |
| | | [0.73, 0.88] | [0.72, 0.91] | [0.68, 0.9] | [0.47, 0.73] | |
| RAB8B | avNNet | 0.86 | 0.76 | 0.8 | 0.56 | 0.482 |
| | | [0.78, 0.93] | [0.66, 0.86] | [0.71, 0.89] | [0.44, 0.69] | |
| SNORA38_5 | svmRadial | 0.84 | 0.81 | 0.76 | 0.57 | 0.482 |
| | | [0.76, 0.92] | [0.72, 0.91] | [0.67, 0.86] | [0.45, 0.7] | |
| ARHGAP29 | c50 | 0.82 | 0.82 | 0.78 | 0.59 | 0.481 |
| | | [0.76, 0.88] | [0.73, 0.91] | [0.68, 0.87] | [0.47, 0.7] | |
| CD68 | rfeLR | 0.82 | 0.8 | 0.79 | 0.59 | 0.479 |
| | | [0.78, 0.85] | [0.76, 0.84] | [0.74, 0.83] | [0.52, 0.65] | |
| NOMO1 | avNNet | 0.85 | 0.81 | 0.75 | 0.56 | 0.478 |
| | | [0.79, 0.91] | [0.73, 0.9] | [0.67, 0.83] | [0.45, 0.67] | |
| EPHA4 | svmRadial | 0.85 | 0.76 | 0.8 | 0.56 | 0.478 |
| | | [0.78, 0.92] | [0.67, 0.86] | [0.72, 0.88] | [0.44, 0.68] | |
| CNN3 | avNNet | 0.85 | 0.82 | 0.74 | 0.56 | 0.478 |
| | | [0.78, 0.92] | [0.75, 0.9] | [0.64, 0.84] | [0.44, 0.68] | |
| PDGFRB | svmRadial | 0.85 | 0.82 | 0.74 | 0.56 | 0.478 |
| | | [0.79, 0.91] | [0.74, 0.91] | [0.64, 0.83] | [0.45, 0.68] | |
| SH3D19 | svmRadial | 0.83 | 0.8 | 0.78 | 0.57 | 0.478 |
| | | [0.76, 0.9] | [0.71, 0.89] | [0.69, 0.86] | [0.45, 0.7] | |
| GK | rfeLR | 0.84 | 0.78 | 0.79 | 0.56 | 0.475 |
| | | [0.81, 0.88] | [0.73, 0.82] | [0.74, 0.83] | [0.5, 0.62] | |
| MIOS | svmRadial | 0.84 | 0.79 | 0.78 | 0.56 | 0.475 |
| | | [0.78, 0.91] | [0.71, 0.87] | [0.67, 0.88] | [0.44, 0.69] | |
| UGCG | rfeLR | 0.86 | 0.78 | 0.78 | 0.55 | 0.474 |
| | | [0.83, 0.89] | [0.74, 0.81] | [0.73, 0.82] | [0.49, 0.61] | |
| TLR2 | rfeLR | 0.86 | 0.75 | 0.8 | 0.55 | 0.474 |
| | | [0.83, 0.89] | [0.69, 0.81] | [0.75, 0.85] | [0.48, 0.62] | |
| DSC2 | c50 | 0.86 | 0.76 | 0.79 | 0.55 | 0.474 |
| | | [0.79, 0.93] | [0.67, 0.86] | [0.7, 0.88] | [0.42, 0.68] | |
| AKT1 | svmRadial | 0.86 | 0.74 | 0.81 | 0.55 | 0.474 |
| | | [0.8, 0.92] | [0.63, 0.85] | [0.73, 0.9] | [0.42, 0.68] | |
| CXCR2P1 | svmRadial | 0.81 | 0.88 | 0.71 | 0.59 | 0.474 |
| | | [0.72, 0.89] | [0.8, 0.95] | [0.61, 0.81] | [0.46, 0.71] | |
| TGFBR2 | avNNet | 0.88 | 0.75 | 0.79 | 0.54 | 0.474 |
| | | [0.81, 0.96] | [0.64, 0.86] | [0.69, 0.89] | [0.41, 0.66] | |
| FTO | svmRadial | 0.88 | 0.79 | 0.75 | 0.54 | 0.474 |
| | | [0.82, 0.94] | [0.71, 0.87] | [0.64, 0.86] | [0.43, 0.65] | |
| TMEM136 | avNNet | 0.9 | 0.84 | 0.69 | 0.52 | 0.473 |
| | | [0.85, 0.95] | [0.76, 0.91] | [0.58, 0.79] | [0.39, 0.66] | |
| PLEKHO1 | svmRadial | 0.84 | 0.75 | 0.81 | 0.56 | 0.471 |
| | | [0.77, 0.91] | [0.66, 0.84] | [0.73, 0.89] | [0.44, 0.68] | |
| VWF | rfeLR | 0.86 | 0.78 | 0.78 | 0.55 | 0.471 |
| | | [0.82, 0.89] | [0.73, 0.82] | [0.73, 0.82] | [0.49, 0.61] | |
| ZFAND5 | rfeLR | 0.82 | 0.81 | 0.76 | 0.57 | 0.471 |
| | | [0.78, 0.86] | [0.76, 0.86] | [0.72, 0.81] | [0.5, 0.65] | |
| BAIAP2_AS1 | rfeLR | 0.82 | 0.76 | 0.81 | 0.57 | 0.471 |
| | | [0.77, 0.87] | [0.71, 0.82] | [0.77, 0.86] | [0.51, 0.64] | |
| GLRX5 | avNNet | 0.82 | 0.79 | 0.79 | 0.57 | 0.470 |
| | | [0.75, 0.89] | [0.69, 0.88] | [0.69, 0.88] | [0.44, 0.7] | |
| DEK | rfeLR | 0.8 | 0.76 | 0.82 | 0.59 | 0.470 |
| | | [0.76, 0.84] | [0.72, 0.81] | [0.78, 0.87] | [0.53, 0.65] | |
| C1orf123 | svmRadial | 0.79 | 0.83 | 0.76 | 0.59 | 0.469 |
| | | [0.69, 0.9] | [0.75, 0.92] | [0.65, 0.87] | [0.45, 0.73] | |
| RNF180 | avNNet | 0.83 | 0.74 | 0.82 | 0.56 | 0.468 |
| | | [0.75, 0.91] | [0.63, 0.85] | [0.73, 0.92] | [0.43, 0.7] | |
| HK3 | avNNet | 0.83 | 0.71 | 0.85 | 0.56 | 0.468 |
| | | [0.76, 0.9] | [0.6, 0.82] | [0.76, 0.94] | [0.44, 0.68] | |
| ZUFSP | rfeLR | 0.85 | 0.79 | 0.76 | 0.55 | 0.468 |
| | | [0.81, 0.89] | [0.73, 0.84] | [0.7, 0.82] | [0.47, 0.63] | |
| IBSP | svmRadial | 0.85 | 0.72 | 0.82 | 0.55 | 0.468 |
| | | [0.78, 0.92] | [0.63, 0.82] | [0.74, 0.91] | [0.43, 0.67] | |
| IRAK3 | rfeLR | 0.81 | 0.75 | 0.82 | 0.57 | 0.467 |
| | | [0.77, 0.86] | [0.7, 0.8] | [0.77, 0.88] | [0.51, 0.64] | |
| IL13 | svmRadial | 0.78 | 0.82 | 0.78 | 0.6 | 0.465 |
| | | [0.69, 0.86] | [0.74, 0.91] | [0.69, 0.86] | [0.48, 0.72] | |
| PIK3CB | avNNet | 0.82 | 0.79 | 0.78 | 0.56 | 0.464 |
| | | [0.75, 0.9] | [0.69, 0.89] | [0.69, 0.86] | [0.41, 0.71] | |
| LUCAT1 | svmRadial | 0.84 | 0.81 | 0.74 | 0.55 | 0.464 |
| | | [0.77, 0.92] | [0.73, 0.9] | [0.65, 0.83] | [0.44, 0.66] | |
| ASS1 | avNNet | 0.79 | 0.9 | 0.69 | 0.59 | 0.463 |
| | | [0.71, 0.87] | [0.83, 0.97] | [0.58, 0.79] | [0.46, 0.71] | |
| CTPS2 | avNNet | 0.83 | 0.77 | 0.79 | 0.55 | 0.458 |
| | | [0.75, 0.92] | [0.66, 0.87] | [0.69, 0.89] | [0.42, 0.68] | |
| ZNF426 | rfeLR | 0.87 | 0.76 | 0.76 | 0.52 | 0.456 |
| | | [0.84, 0.9] | [0.71, 0.82] | [0.71, 0.81] | [0.45, 0.6] | |
| ZCCHC6 | c50 | 0.81 | 0.78 | 0.79 | 0.56 | 0.455 |
| | | [0.72, 0.89] | [0.66, 0.89] | [0.69, 0.88] | [0.42, 0.71] | |
| ARHGAP18 | svmRadial | 0.81 | 0.79 | 0.78 | 0.56 | 0.454 |
| | | [0.72, 0.89] | [0.69, 0.88] | [0.67, 0.88] | [0.42, 0.7] | |
| APOE | c50 | 0.82 | 0.71 | 0.84 | 0.55 | 0.450 |
| | | [0.75, 0.89] | [0.62, 0.81] | [0.74, 0.94] | [0.4, 0.7] | |
| MIR4536_1 | svmRadial | 0.84 | 0.72 | 0.81 | 0.54 | 0.450 |
| | | [0.75, 0.92] | [0.62, 0.83] | [0.72, 0.91] | [0.37, 0.7] | |
| PRG4 | avNNet | 0.84 | 0.79 | 0.75 | 0.54 | 0.450 |
| | | [0.76, 0.91] | [0.7, 0.88] | [0.65, 0.85] | [0.4, 0.68] | |
| CEP170 | svmRadial | 0.8 | 0.75 | 0.81 | 0.56 | 0.450 |
| | | [0.71, 0.89] | [0.64, 0.86] | [0.72, 0.91] | [0.42, 0.7] | |
| PCDHB12 | avNNet | 0.86 | 0.8 | 0.72 | 0.52 | 0.450 |
| | | [0.79, 0.93] | [0.71, 0.89] | [0.63, 0.82] | [0.39, 0.66] | |
| SPARC | rfeLR | 0.78 | 0.85 | 0.72 | 0.57 | 0.449 |
| | | [0.74, 0.83] | [0.81, 0.89] | [0.67, 0.78] | [0.5, 0.65] | |
| IRF8 | rfeLR | 0.88 | 0.75 | 0.76 | 0.51 | 0.448 |
| | | [0.84, 0.91] | [0.7, 0.8] | [0.7, 0.82] | [0.44, 0.58] | |
| CD72 | rfeLR | 0.88 | 0.72 | 0.79 | 0.51 | 0.448 |
| | | [0.85, 0.9] | [0.68, 0.77] | [0.74, 0.83] | [0.46, 0.56] | |
| FABP5P3 | rfeLR | 0.81 | 0.75 | 0.8 | 0.55 | 0.447 |
| | | [0.77, 0.86] | [0.69, 0.81] | [0.75, 0.85] | [0.47, 0.63] | |
| MNDA | svmRadial | 0.81 | 0.85 | 0.7 | 0.55 | 0.447 |
| | | [0.73, 0.89] | [0.78, 0.92] | [0.6, 0.8] | [0.45, 0.65] | |
| LSP1 | svmRadial | 0.83 | 0.74 | 0.8 | 0.54 | 0.447 |
| | | [0.74, 0.92] | [0.64, 0.84] | [0.71, 0.89] | [0.4, 0.68] | |
| SGCB | avNNet | 0.79 | 0.86 | 0.7 | 0.56 | 0.446 |
| | | [0.71, 0.88] | [0.78, 0.94] | [0.59, 0.81] | [0.41, 0.71] | |
| PTP4A3 | avNNet | 0.85 | 0.75 | 0.78 | 0.52 | 0.446 |
| | | [0.78, 0.92] | [0.66, 0.84] | [0.68, 0.87] | [0.4, 0.65] | |
| UBA6 | rfeLR | 0.87 | 0.74 | 0.78 | 0.51 | 0.445 |
| | | [0.83, 0.91] | [0.69, 0.78] | [0.72, 0.83] | [0.43, 0.59] | |
| IL17B | svmRadial | 0.87 | 0.78 | 0.74 | 0.51 | 0.445 |
| | | [0.81, 0.92] | [0.67, 0.88] | [0.64, 0.83] | [0.43, 0.6] | |
| SNORD116_2_1 | avNNet | 0.77 | 0.75 | 0.82 | 0.57 | 0.444 |
| | | [0.69, 0.85] | [0.65, 0.85] | [0.74, 0.91] | [0.44, 0.71] | |
| TRAF5 | svmRadial | 0.89 | 0.7 | 0.8 | 0.5 | 0.444 |
| | | [0.83, 0.94] | [0.59, 0.81] | [0.69, 0.91] | [0.37, 0.63] | |
| FUCA2 | avNNet | 0.81 | 0.82 | 0.72 | 0.55 | 0.443 |
| | | [0.73, 0.88] | [0.74, 0.91] | [0.63, 0.82] | [0.44, 0.66] | |
| 43891.00 | avNNet | 0.81 | 0.85 | 0.7 | 0.55 | 0.443 |
| | | [0.73, 0.88] | [0.77, 0.93] | [0.6, 0.8] | [0.43, 0.67] | |
| FAM35BP | pls | 0.81 | 0.78 | 0.78 | 0.55 | 0.443 |
| | | [0.71, 0.9] | [0.67, 0.88] | [0.67, 0.88] | [0.4, 0.7] | |
| CD83 | svmRadial | 0.81 | 0.78 | 0.78 | 0.55 | 0.443 |
| | | [0.72, 0.89] | [0.68, 0.87] | [0.67, 0.88] | [0.42, 0.68] | |
| MKX | avNNet | 0.82 | 0 8 | 0.74 | 0.54 | 0.443 |
| | | [0.75, 0.9] | [0.71, 0.89] | [0.62, 0.85] | [0.4, 0.67] | |
| BID | svmRadial | 0.84 | 0.72 | 0.8 | 0.52 | 0.443 |
| | | [0.77, 0.92] | [0.64, 0.81] | [0.71, 0.89] | [0.4, 0.65] | |
| MIR718 | svmRadial | 0.84 | 0.69 | 0.84 | 0.52 | 0.443 |
| | | [0.77, 0.92] | [0.58, 0.79] | [0.75, 0.92] | [0.41, 0.64] | |
| FAM126A | svmRadial | 0.86 | 0.81 | 0.7 | 0.51 | 0.442 |
| | | [0.79, 0.93] | [0.73, 0.9] | [0.59, 0.81] | [0.39, 0.64] | |
| FGD6 | pls | 0.77 | 0.76 | 0.81 | 0.57 | 0.441 |
| | | [0.7, 0.84] | [0.67, 0.85] | [0.72, 0.91] | [0.45, 0.7] | |
| DDR1_1 | svmRadial | 0.88 | 0.75 | 0.75 | 0.5 | 0.441 |
| | | [0.82, 0.94] | [0.65, 0.85] | [0.65, 0.85] | [0.37, 0.63] | |
| NFE2L2 | rfeLR | 0.82 | 0.75 | 0.79 | 0.54 | 0.440 |
| | | [0.78, 0.85] | [0.7, 0.8] | [0.74, 0.84] | [0.48, 0.6] | |
| HPRT1 | svmRadial | 0.8 | 0.71 | 0.84 | 0.55 | 0.440 |
| | | [0.7, 0.9] | [0.6, 0.82] | [0.75, 0.92] | [0.4, 0.7] | |
| INSR | avNNet | 0.84 | 0.76 | 0.76 | 0.52 | 0.440 |
| | | [0.77, 0.91] | [0.67, 0.86] | [0.67, 0.85] | [0.39, 0.66] | |
| AKT3 | avNNet | 0.84 | 0.74 | 0.79 | 0.52 | 0.440 |
| | | [0.77, 0.91] | [0.64, 0.84] | [0.69, 0.88] | [0.4, 0.65] | |
| CSF1 | avNNet | 0.81 | 0.86 | 0.69 | 0.54 | 0.439 |
| | | [0.73, 0.89] | [0.79, 0.93] | [0.59, 0.78] | [0.43, 0.66] | |
| TGFBR3 | avNNet | 0.86 | 0.81 | 0.7 | 0.51 | 0.439 |
| | | [0.79, 0.93] | [0.71, 0.91] | [0.59, 0.81] | [0.35, 0.67] | |
| CCND2 | avNNet | 0.83 | 0.75 | 0.78 | 0.52 | 0.436 |
| | | [0.76, 0.9] | [0.65, 0.85] | [0.68, 0.87] | [0.39, 0.66] | |
| ZNF91 | svmRadial | 0.83 | 0.8 | 0.72 | 0.52 | 0.436 |
| | | [0.75, 0.91] | [0.71, 0.89] | [0.64, 0.81] | [0.39, 0.66] | |
| XIAP | svmRadial | 0.77 | 0.68 | 0.9 | 0.57 | 0.435 |
| | | [0.7, 0.84] | [0.57, 0.78] | [0.84, 0.96] | [0.44, 0.69] | |
| ITIH4 | c50 | 0.79 | 0.71 | 0.84 | 0.55 | 0.433 |
| | | [0.72, 0.85] | [0.61, 0.81] | [0.74, 0.94] | [0.42, 0.68] | |
| RBFOX2 | svmRadial | 0.79 | 0.75 | 0.8 | 0.55 | 0.433 |
| | | [0.71, 0.87] | [0.65, 0.85] | [0.7, 0.9] | [0.4, 0.7] | |
| FMN1 | avNNet | 0.82 | 0.64 | 0.89 | 0.52 | 0.433 |
| | | [0.75, 0.9] | [0.53, 0.75] | [0.82, 0.96] | [0.41, 0.64] | |
| TNFSF8 | svmRadial | 0.89 | 0.75 | 0.74 | 0.49 | 0.433 |
| | | [0.83, 0.94] | [0.64, 0.86] | [0.63, 0.85] | [0.36, 0.62] | |
| WDR26 | svmRadial | 0.84 | 0.7 | 0.81 | 0.51 | 0.432 |
| | | [0.78, 0.91] | [0.61, 0.79] | [0.73, 0.9] | [0.39, 0.64] | |
| PTBP3 | rfeLR | 0.84 | 0.7 | 0.81 | 0.51 | 0.432 |
| | | [0.81, 0.88] | [0.66, 0.74] | [0.77, 0.85] | [0.45, 0.57] | |
| FAM35A | c50 | 0.82 | 0.69 | 0.84 | 0.52 | 0.431 |
| | | [0.76, 0.88] | [0.59, 0.78] | [0.75, 0.92] | [0.41, 0.64] | |
| PRPF40A | rfeLR | 0.8 | 0.74 | 0.8 | 0.54 | 0.430 |
| | | [0.76, 0.84] | [0.68, 0.79] | [0.75, 0.85] | [0.48, 0.59] | |
| IL18 | svmRadial | 0.84 | 0.74 | 0.78 | 0.51 | 0.429 |
| | | [0.77, 0.91] | [0.65, 0.83] | [0.69, 0.86] | [0.41, 0.62] | |
| MMP2 | svmRadial | 0.81 | 0.76 | 0.76 | 0.52 | 0.427 |
| | | [0.75, 0.87] | [0.67, 0.86] | [0.65, 0.87] | [0.4, 0.65] | |
| SMARCC2 | avNNet | 0.86 | 0.79 | 0.71 | 0.49 | 0.425 |
| | | [0.8, 0.92] | [0.69, 0.88] | [0.59, 0.83] | [0.35, 0.64] | |
| IL13RA1 | c50 | 0.81 | 0.85 | 0.68 | 0.52 | 0.424 |
| | | [0.73, 0.89] | [0.75, 0.94] | [0.56, 0.79] | [0.38, 0.66] | |
| APOC1 | c50 | 0.79 | 0.74 | 0.8 | 0.54 | 0.423 |
| | | [0.72, 0.86] | [0.64, 0.84] | [0.72, 0.88] | [0.41, 0.67] | |
| HIP1 | svmRadial | 0.81 | 0.81 | 0.71 | 0.52 | 0.423 |
| | | [0.73, 0.89] | [0.72, 0.91] | [0.62, 0.81] | [0.39, 0.66] | |
| IL8 | svmRadial | 0.81 | 0.79 | 0.74 | 0.52 | 0.423 |
| | | [0.72, 0.89] | [0.7, 0.88] | [0.64, 0.84] | [0.38, 0.67] | |
| SNORD97 | svmRadial | 0.86 | 0.75 | 0.74 | 0.49 | 0.423 |
| | | [0.79, 0.93] | [0.66, 0.85] | [0.63, 0.85] | [0.36, 0.63] | |
| CLEC2B | svmRadial | 0.77 | 0.75 | 0.8 | 0.55 | 0.423 |
| | | [0.68, 0.86] | [0.65, 0.85] | [0.7, 0.9] | [0.41, 0.69] | |
| TMOD3 | avNNet | 0.82 | 0.71 | 0.8 | 0.51 | 0.423 |
| | | [0.75, 0.9] | [0.61, 0.81] | [0.71, 0.89] | [0.37, 0.66] | |
| DDR1_5 | rfeLR | 0.82 | 0.76 | 0.76 | 0.51 | 0.422 |
| | | [0.79, 0.85] | [0.71, 0.81] | [0.71, 0.81] | [0.44, 0.58] | |
| SNORA38_3 | svmRadial | 0.86 | 0.74 | 0.75 | 0.49 | 0.420 |
| | | [0.79, 0.93] | [0.64, 0.84] | [0.64, 0.86] | [0.36, 0.61] | |
| SNTB2 | c50 | 0.78 | 0.84 | 0.7 | 0.54 | 0.420 |
| | | [0.71, 0.85] | [0.75, 0.93] | [0.61, 0.79] | [0.4, 0.68] | |
| IGFBP5 | avNNet | 0.84 | 0.72 | 0.78 | 0.5 | 0.419 |
| | | [0.76, 0.91] | [0.62, 0.83] | [0.67, 0.88] | [0.37, 0.63] | |
| C2CD2 | svmRadial | 0.88 | 0.79 | 0.69 | 0.48 | 0.419 |
| | | [0.83, 0.94] | [0.7, 0.88] | [0.57, 0.81] | [0.35, 0.6] | |
| CCL4 | c50 | 0.82 | 0.71 | 0.8 | 0.51 | 0.418 |
| | | [0.74, 0.89] | [0.6, 0.82] | [0.71, 0.89] | [0.38, 0.65] | |
| PDCD11 | avNNet | 0.78 | 0.82 | 0.72 | 0.54 | 0.417 |
| | | [0.68, 0.87] | [0.74, 0.9] | [0.62, 0.83] | [0.42, 0.66] | |
| MIR3153 | svmRadial | 0.81 | 0.76 | 0.75 | 0.51 | 0.416 |
| | | [0.72, 0.91] | [0.67, 0.85] | [0.64, 0.86] | [0.37, 0.65] | |
| KLF8 | avNNet | 0.81 | 0.71 | 0.8 | 0.51 | 0.416 |
| | | [0.74, 0.89] | [0.6, 0.82] | [0.71, 0.89] | [0.38, 0.65] | |
| OGN | svmRadial | 0.83 | 0.78 | 0.72 | 0.5 | 0.416 |
| | | [0.77, 0.89] | [0.69, 0.86] | [0.64, 0.81] | [0.39, 0.61] | |
| CSPG4 | svmRadial | 0.85 | 0.76 | 0.72 | 0.49 | 0.414 |
| | | [0.78, 0.92] | [0.67, 0.85] | [0.62, 0.83] | [0.37, 0.6] | |
| CDKN2A | avNNet | 0.85 | 0.76 | 0.72 | 0.49 | 0.414 |
| | | [0.78, 0.92] | [0.66, 0.86] | [0.61, 0.84] | [0.34, 0.63] | |
| DUSP19 | avNNet | 0.87 | 0.68 | 0.8 | 0.48 | 0.413 |
| | | [0.8, 0.93] | [0.58, 0.77] | [0.71, 0.89] | [0.36, 0.59] | |
| SNAPC1 | avNNet | 0.8 | 0.72 | 0.79 | 0.51 | 0.412 |
| | | [0.73, 0.88] | [0.64, 0.81] | [0.69, 0.89] | [0.37, 0.66] | |
| CTSL1 | avNNet | 0.84 | 0.8 | 0.69 | 0.49 | 0.411 |
| | | [0.77, 0.92] | [0.71, 0.89] | [0.58, 0.79] | [0.35, 0.62] | |
| TGFB2 | avNNet | 0.82 | 0.84 | 0.66 | 0.5 | 0.409 |
| | | [0.75, 0.89] | [0.75, 0.92] | [0.57, 0.75] | [0.37, 0.63] | |
| CTSB | svmRadial | 0.82 | 0.76 | 0.75 | 0.5 | 0.409 |
| | | [0.74, 0.89] | [0.68, 0.85] | [0.65, 0.85] | [0.38, 0.62] | |
| VAMP8 | c50 | 0.76 | 0.75 | 0.79 | 0.54 | 0.408 |
| | | [0.68, 0.84] | [0.67, 0.83] | [0.69, 0.88] | [0.41, 0.67] | |
| ATG3 | glmnet | 0.79 | 0.8 | 0.71 | 0.51 | 0.407 |
| | | [0.71, 0.87] | [0.71, 0.89] | [0.62, 0.81] | [0.38, 0.64] | |
| TANK | svmRadial | 0.81 | 0.71 | 0.79 | 0.5 | 0.403 |
| | | [0.74, 0.88] | [0.61, 0.81] | [0.69, 0.88] | [0.37, 0.63] | |
| FCER1A | avNNet | 0.81 | 0.76 | 0.74 | 0.5 | 0.403 |
| | | [0.73, 0.89] | [0.67, 0.85] | [0.64, 0.83] | [0.36, 0.64] | |
| B3GNT2 | avNNet | 0.81 | 0.72 | 0.78 | 0.5 | 0.403 |
| | | [0.72, 0.89] | [0.62, 0.83] | [0.69, 0.86] | [0.37, 0.63] | |
| FAM134B | svmRadial | 0.76 | 0.78 | 0.75 | 0.52 | 0.400 |
| | | [0.68, 0.85] | [0.69, 0.86] | [0.65, 0.85] | [0.41, 0.64] | |
| NFIL3 | svmRadial | 0.8 | 0.74 | 0.76 | 0.5 | 0.400 |
| | | [0.72, 0.88] | [0.64, 0.83] | [0.66, 0.86] | [0.36, 0.64] | |

The results in this example show that predictions in transcript levels can be extrapolated to specific fundamental processes related to the pathophysiology of atherosclerosis and plaque instability. This information can provide a patient-specific determination of one or more mechanisms related to the subject's plaque pathophysiology, plaque instability, or both. In turn, this data can be used to provide patient-specific therapeutic recommendations, either for specific medications or for specific surgical interventions.

### Example 5: Validation of Predicted Gene Expression from Plaque Morphology

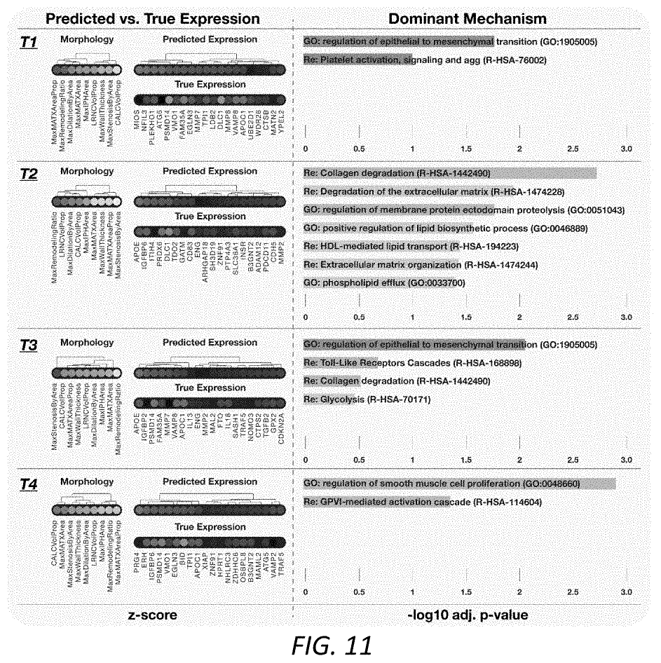

The resulting predictive models were validated by image analysis of CTAs from four patients excluded from model development, but where microarray data from CEA specimens was available for comparison. The plaque morphology heatmaps indicated different plaque characteristics: patient T1 had a lesion with a relatively large proportion of MATX, low CALC and intermediate amount of LRNC and IPH; T2 had high levels of LRNC, low MATX, and intermediate levels of CALC and IPH; while T3 and T4 were more calcified. The predicted 20 most significantly dysregulated transcripts, compared with the true expression of corresponding transcripts, demonstrated unique dominant mechanisms for each patient derived from plaque morphology (FIG. 11; Performance using locked-down models on four sequestered (unseen) test patients (T1-T4). Heatmaps for each test patient representing: plaque morphology profile; predicted expression of the top 20 most significant predicted transcripts; and true expression of corresponding transcripts obtained from microarray analysis of CEA specimens (left column). Dominant mechanisms obtained from pathway analysis by GSEA for each patient (right column)).

Patient T1's profile showed dysregulation of epithelial to mesenchymal transition (adj. p=0.015), while T2's profile resulted in 7 significant processes: collagen degradation (adj. p=0.002), ECM degradation (adj. p=0.011), regulation of membrane protein ectodomain proteolysis (adj. p=0.02), positive regulation of lipid biosynthetic process (adj. p=0.027), HDL-mediated lipid transport (adj. p=0.041), ECM organization (adj. p=0.042), and phospholipid efflux (adj. p=0.043). Patient T3 had significantly dysregulated epithelial to mesenchymal transition (adj. p=0.01). Patient T4 had two significantly dysregulated processes: regulation of SMC proliferation (adj. p=6.2e-04) and GPVI-mediated activation cascade (adj. p=0.047).

In this Example, the validity of the models was tested in a sequestered set of patients by predicting gene expression from plaque morphology by CTA, which was compared with transcriptomic data from corresponding tissue specimens. The results demonstrated a good correlation between predicted and observed expression levels of transcripts, while pathway analysis of the most significant transcripts demonstrated unique dominant mechanisms for each individual. Notably, the analysed plaque of one patient (T2) was dominated by lipid metabolism in a manner quite different from the other patients, which suggests opportunities for patient-specific plaque phenotyping as guidance for individualized therapy.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. World Health Organization (WHO). Cardiovascular diseases (cvds) fact sheet. 2017
2. Bloom D E, Cafiero, E. T., Jané-Llopis, E., Abrahams-Gessel, S., Bloom, L. R., Fathima, S., Feigl, A. B., Gaziano, T., Mowafi, M., Pandya, A., Prettner, K., Rosenberg, L., Seligman, B., Stein, A. Z., Weinstein, C. The global economic burden of noncommunicable diseases. 2011
3. Bergstrom G, Berglund Blomberg A, Brandberg J, Engstrom G, Engvall J, Eriksson M, de Faire U, Flinck A, Hansson M G, et al. The Swedish cardiopulmonary bioimage study: Objectives and design. J Intern Med. 2015; 278:645-659
4. Newby D E, Adamson P D, Berry C, Boon N A, Dweck M R, Flather M, Forbes J, Hunter A, Lewis S, MacLean S, et al. Coronary C T angiography and 5-year risk of myocardial infarction. The New England journal of medicine. 2018; 379:924-933
5. Lyngbakken M N, Myhre P L, Rosjo H, Omland T. Novel biomarkers of cardiovascular disease: Applications in clinical practice. Crit Rev Clin Lab Sci. 2019; 56:33-60
6. Saba L, Saam T, Jäger H R, Yuan C, Hatsukami T S, Saloner D, Wasserman B A, Bonati L H, Wintermark M. Imaging biomarkers of vulnerable carotid plaques for stroke risk prediction and their potential clinical implications. Lancet Neurol. 2019; 18:559-572
7. Hafiane A. Vulnerable plaque, characteristics, detection, and potential therapies. J Cardiovasc Dev Dis. 2019; 6
8. Taking personalized medicine to heart. Nat Med. 2018; 24:113
9. Macklin P. Key challenges facing data-driven multicellular systems biology. arXiv preprint arXiv:1806.04736. 2018
10. Bergmann F T, Hoops S, Klahn B, Kummer U, Mendes P, Pahle J, Sahle S. Copasi and its applications in biotechnology. J Biotechnol. 2017; 261:215-220
11. Pârvu O, Gilbert D. A novel method to verify multilevel computational models of biological systems using multiscale spatio-temporal meta model checking. PloS one. 2016; 11:e0154847
12. Sorger P K. Quantitative and systems pharmacology in the postgenomic era: New approaches to discovering drugs and understanding therapeutic mechanisms. 2011.
13. Okuda S, Yamada T, Hamajima M, Itoh M, Katayama T, Bork P, Goto S, Kanehisa M. Kegg atlas mapping for global analysis of metabolic pathways. Nucleic Acids Res. 2008; 36:W423-426
14. Altafini C. Odes models in systems biology. 2007
15. Kholodenko B N. Cell-signalling dynamics in time and space. Nat Rev Mol Cell Biol. 2006; 7:165-176
16. Eungdamrong N J, Iyengar R. Modeling cell signaling networks. Biol Cell. 2004; 96:355-362
17. Gomez-Cabrero D, Compte A, Tegner J. Workflow for generating competing hypothesis from models with parameter uncertainty. Interface Focus. 2011; 1:438-449
18. Dejana E, Hirschi K K, Simons M. The molecular basis of endothelial cell plasticity. Nat Commun. 2017; 8:14361
19. Chappell J, Harman J L, Narasimhan V M, Yu H, Foote K, Simons B D, Bennett M R, Jorgensen H F. Extensive proliferation of a subset of differentiated, yet plastic, medial vascular smooth muscle cells contributes to neointimal formation in mouse injury and atherosclerosis models. Circ Res. 2016; 119:1313-1323
20. Lin M-E, Chen T M, Wallingford M C, Nguyen N B, Yamada S, Sawangmake C, Zhang J, Speer M Y, Giachelli C M. Runx2 deletion in smooth muscle cells inhibits vascular osteochondrogenesis and calcification but not atherosclerotic lesion formation. Cardiovascular research. 2016; 112:606-616
21. Kitada M, Ogura Y, Koya D. The protective role of sirt1 in vascular tissue: Its relationship to vascular aging and atherosclerosis. Aging (Albany N.Y.). 2016; 8:2290-2307
22. Cherepanova O A, Gomez D, Shankman L S, Swiatlowska P, Williams J, Sarmento O F, Alencar G F, Hess D L, Bevard M H, Greene E S, et al. Activation of the pluripotency factor oct4 in smooth muscle cells is atheroprotective. Nat Med. 2016; 22:657-665
23. Shankman L S, Gomez D, Cherepanova O A, Salmon M, Alencar G F, Haskins R M, Swiatlowska P, Newman A A, Greene E S, Straub A C. Klf4-dependent phenotypic modulation of smooth muscle cells has a key role in atherosclerotic plaque pathogenesis. Nature medicine. 2015; 21:628
24. Nording H M, Seizer P, Langer H F. Platelets in inflammation and atherogenesis. Front Immunol. 2015; 6:98
25. Fenning R S, Burgert M E, Hamamdzic D, Peyster E Q Mohler E R, Kangovi S, Jucker B M, Lenhard S C, Macphee C H, Wilensky R L. Atherosclerotic plaque inflammation varies between vascular sites and correlates with response to inhibition of lipoprotein-associated phospholipase a2. Journal of the American Heart Association. 2015; 4:e001477
26. Lambin P, Leijenaar R T H, Deist T M, Peerlings J, de Jong E E C, van Timmeren J, Sanduleanu S, Lame R T H M, Even A J G; Jochems A, et al. Radiomics: The bridge between medical imaging and personalized medicine. Nature Reviews Clinical Oncology. 2017; 14:749-762
27. Lee Lee H Y, Ko E S, Jeong W K. Radiomics and imaging genomics in precision medicine. Precision and Future Medicine. 2017; 1:10-31
28. Campbell B C V, De Silva D A, Macleod M R, Coutts S B, Schwamm L H, Davis S M, Donnan G A. Ischaemic stroke. Nat Rev Dis Primers. 2019; 5:70
29. Ibrahimi P, Jashari F, Nicoll R, Bajraktari Wester P, Henein M Y. Coronary and carotid atherosclerosis: How useful is the imaging? Atherosclerosis.231:323-333
30. Cornelissen A, Jinnouchi H, Sakamoto A, Torii S, Kuntz S, Guo L, Fernandez R, Paek K, Mayhew C, Kutyna M, et al. Evaluation and management of the vulnerable plaque. Current Cardiovascular Risk Reports. 2019; 13:14
31. King J Y, Ferrara R, Tabibiazar R, Spin J M, Chen M M, Kuchinsky A, Vailaya A, Kincaid R, Tsalenko A, Deng D X, et al. Pathway analysis of coronary atherosclerosis. Physiol Genomics. 2005; 23:103-118
32. Choi H, Uceda D E, Dey A K, Abdelrahman K M, Aksentijevich M, Rodante J A, Elnabawi Y A, Reddy A, Keel A, Erb-Alvarez J, et al. Treatment of psoriasis with biologic therapy is associated with improvement of coronary artery plaque lipid-rich necrotic core: Results from a prospective, observational study. Circulation. Cardiovascular imaging. 2020; 13:e011199
33. Matic L P, Jesus Iglesias M, Vesterlund M, Lengquist M, Hong M Q Saieed S, Sanchez-Rivera L, Berg M, Razuvaev A, Kronqvist M, et al. Novel multiomics profiling of human carotid atherosclerotic plaques and plasma reveals biliverdin reductase b as a marker of intraplaque hemorrhage. JACC Basic Transl Sci. 2018; 3:464-480
34. Sheahan M, Ma X, Paik D, Obuchowski N A, St Pierre S, Newman W P, 3rd, Rae Perlman E S, Rosol M, Keith J C, Jr., et al. Atherosclerotic plaque tissue: Noninvasive quantitative assessment of characteristics with software-aided measurements from conventional ct angiography. Radiology. 2018; 286:622-631
35. MacRae C A, Califf R M. Reimagining what we measure in atherosclerosis-a “phenotype stack”. Circ Res. 2020; 126:1146-1158
36. North American Symptomatic Carotid Endarterectomy Trial C, Barnett H J M, Taylor D W, Haynes R B, Sackett D L, Peerless S J, Ferguson G G Fox A J, Rankin R N, Hachinski V C, et al. Beneficial effect of carotid endarterectomy in symptomatic patients with high-grade carotid stenosis. The New England journal of medicine. 1991; 325:445-453
37. Karla E, Seime T, Dias N, Lengquist M, Witasp A, Almqvist H, Kronqvist M, Gådin J R, Odeberg J, Maegdefessel L, et al. Correlation of computed tomography with carotid plaque transcriptomes associates calcification with lesion-stabilization. Atherosclerosis. 2019; 288:175-185
38. Perisic L, Aldi S, Sun Y, Folkersen L, Razuvaev A, Roy J, Lengquist M, Åkesson S, Wheelock C E, Maegdefessel L, et al. Gene expression signatures, pathways and networks in carotid atherosclerosis. J Intern Med. 2016; 279:293-308
39. Perisic Matic L, Rykaczewska U, Razuvaev A, Sabater-Lleal M, Lengquist M, Miller C L, Ericsson I, Rohl S, Kronqvist M, Aldi S, et al. Phenotypic modulation of smooth muscle cells in atherosclerosis is associated with downregulation of lmod1, synpo2, pdlim7, pin, and synm. Arteriosclerosis, thrombosis, and vascular biology. 2016; 36:1947-1961
40. Abdelrahman K M, Chen M Y, Dey A K, Virmani R, Finn A V, Khamis R Y, Choi A D, Min J K, Williams M C, Buckler A J, et al. Coronary computed tomography angiography from clinical uses to emerging technologies: JACC state-of-the-art review. Journal of the American College of Cardiology. 2020; 76:1226-1243
41. van Assen M, Varga-Szemes A, Egorova S, Johnson K, St. Pierre S, Zaki B, Schoepf U J, Buckler A J. Automated plaque analysis for the prognostication of major adverse cardiac events. European Society of Cardiology. 2019; 116:8
42. Zhu Li Y, Ding V, et al. Semiautomated Characterization of Carotid Artery Plaque Features From Computed Tomography Angiography to Predict Atherosclerotic Cardiovascular Disease Risk Score. J Comput Assist Tomogr. 2019; 43(3):452-459. doi:10.1097/RCT.0000000000000862
43. Rafailidis V, Chryssogonidis I, Xerras C, Tegos T, Nikolaou I, Charitanti-Kouridou A, Destanis E, Kalogera-Fountzila A. Carotid plaque vulnerability: The correlation of plaque components as quantified based on computed tomography angiography with neurologic symptoms. European Congress of Radiology. 2019, Poster Number: C-0161
44. Chrencik M T, Khan A A, Luther L, Anthony L, Yokemick J, Patel J, Sorkin J D, Sikdar S, Lal B K. Quantitative assessment of carotid plaque morphology (geometry and tissue composition) using computed tomography angiography. Journal of Vascular Surgery. 2019
45. Buckler A. 510(k) k183012. 2018
46. Hopkins P N. Molecular biology of atherosclerosis. Physiol Rev. 2013; 93:1317-1542
47. Rudin C. Stop explaining black box machine learning models for high stakes decisions and use interpretable models instead. Nature Machine Intelligence. 2019; 1:206-215
48. Kuhn M, Johnson K. Applied predictive modeling. New York: Springer; 2013.
49. Brinjikji W, Huston J, Rabinstein A A, Kim G M, Lerman A, Lanzino G Contemporary carotid imaging: From degree of stenosis to plaque vulnerability. J Neurosurg. 2016; 124:27-42
50. Shah P K. Inflammation and plaque vulnerability. Cardiovasc Drugs Ther. 2009; 23:31-40
51. Ahmadi A, Leipsic J, Øvrehus K A, Gaur S, Bagiella E, Ko B, Dey D, LaRocca G, Jensen J M, Bøtker H E, et al. Lesion-specific and vessel-related determinants of fractional flow reserve beyond coronary artery stenosis. JACC. Cardiovascular imaging. 2018; 11:521-530
52. Zalewski P D, Beltrame J F, Wawer A A, Abdo A I, Murgia C. Roles for endothelial zinc homeostasis in vascular physiology and coronary artery disease. Crit Rev Food Sci Nutr. 2019; 59:3511-3525
53. Abdelrahman K, Virmani R, Buckler A, Mehta N. Coronary computed tomography angiography: From clinical uses to emerging technologies. JACC. 2020; in press
54. Holdt L M, Sass K, Gabel Bergert H, Thiery J, Teupser D. Expression of chr9p21 genes cdkn2b (p15(ink4b)), cdkn2a (p16(ink4a), p14(arf)) and mtap in human atherosclerotic plaque. Atherosclerosis. 2011; 214:264-270
55. Naylor A, Rothwell P, Bell P. Overview of the principal results and secondary analyses from the european and north american randomised trials of endarterectomy for symptomatic carotid stenosis. European Journal of Vascular and Endovascular Surgery. 2003; 26:115-129
56. Qamar A, Rader D J. Effect of interleukin 1beta inhibition in cardiovascular disease. Curr Opin Lipidol. 2012; 23:548-553
57. Alexander M R, Moehle C W, Johnson J L, Yang Z, Lee J K, Jackson C L, Owens G K. Genetic inactivation of il-1 signaling enhances atherosclerotic plaque instability and reduces outward vessel remodeling in advanced atherosclerosis in mice. J Clin Invest. 2012; 122:70-79
58. Libby P. Interleukin-1 beta as a target for atherosclerosis therapy: Biological basis of cantos and beyond. Journal of the American College of Cardiology. 2017; 70:2278-2289
59. Mandessian H, Perisic Matic L, Lengquist M, Gertow K, Sennblad B, Baldassarre D, Veglia F, Humphries S E, Rauramaa R, de Faire U, et al. Integrative studies implicate matrix metalloproteinase-12 as a culprit gene for large-artery atherosclerotic stroke. J Intern Med. 2017; 282:429-444
60. Shi X, Gao J, Lv Q, Cai H, Wang F, Ye R, Liu X. Calcification in atherosclerotic plaque vulnerability: Friend or foe? Frontiers in physiology. 2020; 11:56-56
61. Lu Y, Thavarajah T, Gu W, Cai J, Xu Q. Impact of mirna in atherosclerosis. Arteriosclerosis, thrombosis, and vascular biology. 2018; 38:e159-e170
62. Nanoudis S, Pikilidou M, Yavropoulou M, Zebekakis P. The role of micrornas in arterial stiffness and arterial calcification. An update and review of the literature. Front Genet. 2017; 8:209
63. Eken S M, Jin H, Chernogubova E, Li Y, Simon N, Sun C, Korzunowicz Busch A, Bäcklund A, Österholm C, et al. Microrna-210 enhances fibrous cap stability in advanced atherosclerotic lesions. Circ Res. 2017; 120:633-644
64. Li X, Yao N, Zhang J, Liu Z. Microrna-125b is involved in atherosclerosis obliterans in vitro by targeting podocalyxin. Mol Med Rep. 2015; 12:561-568
65. Gozuacik D, Akkoc Y, Ozturk D G, Kocak M. Autophagy-regulating micrornas and cancer. Front Oncol. 2017; 7:65
66. Grootaert M O J, Roth L, Schrijvers D M, De Meyer G R Y, Martinet W. Defective autophagy in atherosclerosis: To die or to senesce? Oxid Med Cell Longev. 2018; 2018:7687083
67. Zavaczki E, Gall T, Zarjou A, Hendrik Z, Potor L, Toth C Z, Mehes Gyetvai A, Agarwal A, Balla et al. Ferryl hemoglobin inhibits osteoclastic differentiation of macrophages in hemorrhaged atherosclerotic plaques. Oxid Med Cell Longev. 2020; 2020:3721383
68. Sabatine M S. Pcsk9 inhibitors: Clinical evidence and implementation. Nature reviews. Cardiology. 2019; 16:155-165
69. Buckler A J, Bresolin L, Dunnick N R, Sullivan D C, Aerts H J, Bendriem B, Bendtsen C, Boellaard R, Boone J M, Cole P E, et al. Quantitative imaging test approval and biomarker qualification: Interrelated but distinct activities. Radiology. 2011; 259:875-884

What is claimed is:

1. A method of generating phenotypic data for an atherosclerotic plaque from a subject, the method comprising:
(a) receiving a non-invasively obtained imaging dataset for an atherosclerotic plaque from a subject;
(b) processing the non-invasively obtained imaging dataset with a virtual tissue model to obtain quantitative plaque morphology data;
(c) processing the quantitative plaque morphology data with a virtual expression model to obtain estimated gene expression data for the plaque from the subject; and
(d) predicting which gene transcript levels are elevated and which gene levels are decreased in the plaque from the subject as compared to gene expression in a subject without atherosclerosis, thereby generating phenotypic data for the atherosclerotic plaque from the subject.

2. The method of claim 1, wherein the non-invasively obtained imaging dataset is a radiological imaging dataset.

3. The method of claim 2, wherein the non-invasively obtained radiological imaging dataset is obtained by computed tomography (CT), dual energy computed tomography (DECT), spectral computed tomography (spectral CT), computed tomography angiography (CTA), cardiac computed tomography angiography (CCTA), magnetic resonance imaging (MRI), multi-contrast magnetic resonance imaging (multi-contrast MRI), ultrasound (US), positron emission tomography (PET), intra-vascular ultrasound (IVUS), optical coherence tomography (OCT), near-infrared radiation spectroscopy (NIRS), or single-photon emission tomography (SPECT) diagnostic images or any combination thereof.

4. The method of claim 1, wherein quantitative plaque morphology data comprises structural anatomy data and tissue composition data.

5. The method of claim 4, wherein the structural anatomy data comprises data relating to a level of any one or more of remodeling, wall thickening, ulceration, stenosis, dilation, or plaque burden.

6. The method of claim 4, wherein the tissue composition data comprises data relating to a level of any one or more of calcification, lipid-rich necrotic core (LRNC), intraplaque hemorrhage (IPH), matrix, fibrous cap, or perivascular adipose tissue (PVAT).

7. The method of claim 1, wherein the gene transcript levels are based on gene transcripts whose expression profiles are illustrated in FIG. 5.

8. The method of claim 1, wherein the gene transcript levels are based on gene transcripts listed in Table 4, gene transcripts listed in Table 5, or gene transcripts listed in both Table 4 and Table 5.

9. The method of claim 1, further comprising using the predicted gene transcript levels for gene-set enrichment analysis to provide a patient-specific determination of one or more mechanisms related to the subject's plaque pathophysiology, plaque instability, or both.

10. The method of claim 9, wherein the one or more mechanisms related to plaque pathophysiology, plaque instability, or both comprise one or more of smooth muscle cell (SMC) proliferation, extracellular matrix (ECM) organization, collagen degradation, phospholipid efflux, degradation of the extracellular matrix, positive regulation of intracellular signal transduction, regulation of epithelial to mesenchymal transition, regulation of IGF transport and uptake, homotypic cell-cell adhesion, neutrophil mediated immunity, apoptotic process, regulation of protein ectodomain proteolysis, cholesterol efflux, chylomicron remnant clearance, response to laminar fluid shear stress, or neutrophil mediated immunity.

11. The method of claim 1, wherein imaging data intensity is corrected to more closely represent the originally imaged plaque using a patient-specific three-dimensional point spread function.

12. The method of claim 1, wherein the virtual expression model comprises a supervised continuous gene expression model or a dichotomized gene expression model of gene expression levels above or below a median expression value.

13. The method of claim 1, wherein a plaque classified as having a high level of calcification compared to a reference level is predicted to have a high level of expression of proteoglycan 4, a low level of expression of Speedy/RINGO Cell Cycle Regulator Family Member E1, a low level of expression of Solute Carrier Family 30 Member 1, and a low level of expression of Solute Carrier Family 39 Member 8, as compared to corresponding reference levels of expression in a plaque that does not have a high level of calcification.

14. The method of claim 1, wherein a plaque classified as having a large LRNC compared to a reference level is predicted to have a high level of expression of matrix metalloproteinase 12, a high level of expression of Solute Carrier Family 39 Member 8, a high level of expression of IL1R1, a low level of expression of rap guanine nucleotide exchange factor 4, and a low level of expression of Solute Carrier Family 30 Member 1, as compared to corresponding reference levels of expression in a plaque that does not have a large LRNC.

15. The method of claim 1, wherein a plaque classified as having a high level of IPH compared to a reference level is predicted to have a higher level of expression of biliverdin reductase B, a high level of expression of cyclin-dependent kinase inhibitor 2A, a high level of expression of Solute Carrier Family 30 Member 1, a high level of expression of Solute Carrier Family 39 Member 8, and a low level of expression of nodal modulator 1 as compared to corresponding reference levels of expression in a plaque that does not have a high level of IPH.

16. The method of claim 1, wherein a plaque classified as having high level of calcification compared to a reference level and low level of IPH compared to a reference level is predicted to have high level of expression of TGFBR2 as compared to a corresponding reference level of expression in a plaque that does not have a high level of calcification and a low level of IPH.

17. The method of claim 1, wherein a plaque classified as having a large amount of matrix compared to a reference level is predicted to have high level of expression of interleukin-13 and a low level of expression of Nudix Hydrolase 21 as compared to corresponding reference levels of expression in a plaque that does not have a large amount of matrix.

18. The method of claim 1, wherein a low level of expression of MIR125B1 compared to a reference level is predicted in a plaque with a combined large LRNC and a high level of IPH, compared to a reference level, and a high level of expression pf MIR125B1 compared to a reference level is predicted in a small plaque with a high level of CALC compared to a corresponding reference level.

19. The method of claim 1, wherein a low level of expression of MIR718 compared to a reference level is predicted in a small plaque with a high level of CALC, compared to a reference level, and a level of expression of MIR718 is increased in a larger plaque as a level of CALC decreases compared to a corresponding reference level.

20. The method of claim 1, wherein a level of expression of MIR4536-1 is predicted to be lower, compared to a corresponding reference level, in a large plaque with an increased level of CALC, compared to a corresponding reference level, and is predicted to be even lower in a plaque with a decreased level of CALC.
